SEQUENCE LISTING (20 sequences)

SEQ ID NO: 1 - protein, 8 aa, Hepatitis C virus
  misc_feature (1)..(1): Xaa may be Met or Lys
XXXKXXXX

SEQ ID NO: 2 - DNA, 25 bp, Hepatitis C virus
  modified_base (1)..(25): Residues may be modified
atggagacyaagvtyatyacstggg

SEQ ID NO: 3 - protein, 10 aa, Hepatitis C virus
  misc_feature (4)..(4): Xaa may be Gly or Cys
STYXKFLADG

SEQ ID NO: 4 - DNA, 29 bp, Hepatitis C virus
  modified_base (1)..(29): Residues may be modified
ccgtcggcaagraacttgccrtaggtgga

SEQ ID NO: 5 - protein, 10 aa, Hepatitis C virus
  misc_feature (2)..(2): Xaa may be Pro or His
AXITAYXXQT

SEQ ID NO: 6 - DNA, 29 bp, Hepatitis C virus
  modified_base (1)..(29): Residues may be modified
gcgccyatyacggcctaykcccarcarac

SEQ ID NO: 7 - DNA, 52 bp, Hepatitis C virus
  modified_base (1)..(52): Residues may be modified
agggcatttaaatagccaccatggcgccyatyacggcctaykcccarcarac

SEQ ID NO: 8 - DNA, 48 bp, Hepatitis C virus
  modified_base (1)..(48): Residues may be modified
aaaaaggcgcgccaccatggcgccyatyacggcctaykcccarcarac

SEQ ID NO: 9 - protein, 11 aa, Hepatitis C virus
  misc_feature (3)..(3): Xaa may be Gly or Arg
GSXKXXXVPXX

SEQ ID NO: 10 - DNA, 32 bp, Hepatitis C virus
  modified_base (1)..(32): Residues may be modified
gcagccggcacyttrgtgctyttrccgctrcc

SEQ ID NO: 11 - DNA, 43 bp, Hepatitis C virus
  modified_base (1)..(43): Residues may be modified
aaaaagcggccgcagccggcacyttrgtgctyttrccgctrcc

SEQ ID NO: 12 - DNA, 46 bp, Hepatitis C virus
  modified_base (1)..(46): Residues may be modified
cttggttaattaatgcagccggcacyttrgtgctyttrccgctrcc

SEQ ID NO: 13 - DNA, 25 bp, Hepatitis C virus
atggagaccaagctcatcacgtggg

SEQ ID NO: 14 - DNA, 28 bp, Hepatitis C virus
acccgccgtcggcaaggaacttgccgta

SEQ ID NO: 15 - DNA, 52 bp, Hepatitis C virus
agggcatttaaatagccaccatggcgcccatcacggcgtacgcccagcagac

SEQ ID NO: 16 - DNA, 43 bp, Hepatitis C virus
aaaaagcggccgcagccgggaccttggtgctcttaccgctgcc

SEQ ID NO: 17 - DNA, 25 bp, Hepatitis C virus
atggagaccaagatcatcacctggg

SEQ ID NO: 18 - DNA, 29 bp, Hepatitis C virus
ccgtcggcaaggaacttgccataggtgga

SEQ ID NO: 19 - DNA, 52 bp, Hepatitis C virus
agggcatttaaatagccaccatggcgcccatcacggcctactcccaacagac

SEQ ID NO: 20 - DNA, 43 bp, Hepatitis C virus
aaaaagcggccgcagccggcaccttagtgctcttgccgctgcc

(19) United States
(12) Patent Application Publication
(10) Pub. No.: US 2009/0220943 A1
(43) Pub. Date: Sep. 3, 2009

(54) HCV GENOTYPING AND PHENOTYPING

(75) Inventors: Jin Hong, Brisbane, CA (US); Scott Seiwert, Pacifica, CA (US); Sharlene Lim, San Mateo, CA (US); Xiaoli Qin, San Mateo, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: INTERMUNE, INC., Brisbane, CA (US)

(21) Appl. No.: 12/262,111

(22) Filed: Oct. 30, 2008

Related U.S. Application Data
(60) Provisional application No. 60/983,854, filed on Oct. 30, 2007.
(60) Provisional application No. 61/043,020, filed on Apr. 7, 2008.

(51) Int. Cl. C12Q 1/70; C07H 21/00; C12P 19/34; C12N 15/63

(52) U.S. Cl. 435/5; 536/24.33; 435/91.2; 435/320.1

(57) ABSTRACT

The present invention includes methods of genotyping and phenotyping HCV. In one embodiment, the methods of the invention can be used to determine whether a HCV isolate is resistant to an antiviral drug. The invention also includes primers for amplifying a HCV NS3 region and kits.

The present application claims priority to U.S. Provisional Application No. 60/983,854, filed Oct. 30, 2007, and U.S. Provisional Application No. 61/043,020, filed on Apr. 7, 2008, both of which are incorporated by reference herein in their entirety.

### FIELD OF THE INVENTION

The present invention relates to HCV genotyping and phenotyping assays. The invention includes, for instance, compositions and primers for amplifying an NS3 protease domain of HCV.

### BACKGROUND OF THE INVENTION

Hepatitis C virus (HCV) is an enveloped positive strand RNA virus of the Flaviviridae family. The single strand HCV RNA genome is of positive polarity and comprises one open reading frame of approximately 9600 nucleotides in length, which encodes a polyprotein of approximately 3,010 amino acids. In infected cells, the polyprotein is cleaved at multiple sites by host and viral proteases to produce viral structural and non-structural (NS) proteins.

The structural proteins (C, E1, E2/NS1) make up the nucleocapsid protein and one or two membrane-associated glycoproteins. The non-structural proteins (NS2, NS3, NS4A, NS4B, NS5A and NS5B) are enzymes or accessory factors that catalyze and regulate the replication of the HCV RNA genome. NS3 is both a proteolytic cleavage enzyme and a helicase, to facilitate unwinding of the viral genome for replication. NS5b is an RNA-dependent RNA polymerase needed for viral replication.

HCV affects approximately 3% of the world's population (170 million people) and has been declared by the World Health Organization as a global health epidemic. About 50% to 80% of those infected with HCV develop chronic hepatitis with viral persistence. Subjects with chronic hepatitis are at increased risk for developing liver cirrhosis and hepatocellular carcinoma. End-stage liver disease caused by HCV accounts for approximately 30% to 40% of liver transplants.

The current gold standard for treatment of HCV is pegylated α-interferon in combination with ribavirin, a broad-spectrum antiviral agent. However, the regimen is prolonged and not well tolerated. Further, only approximately half of genotype 1 HCV-infected individuals have a sustained response to the treatment.

There is a great demand for therapeutics that are efficacious and safe alternatives to interferon or that may be used in interferon-containing regimens. Several HCV antiviral treatments are in clinical trials. These therapeutics include, for instance, viral protease inhibitors and polymerase inhibitors. Inhibitors of HCV protease NS3 have been developed which are both highly active and selective. For this reason, these compounds have potential for becoming the next generation of anti-HCV treatment. (See, for instance, WO 00/09543, WO 00/09558 and WO 00/59929, which are each herein incorporated by reference in its entirety.)

Despite progress made in the development of new treatment options for HCV infected patients, drug resistance is a major concern. HCV displays a high genetic diversity that results from defects in the repair activity of RNA-dependent RNA polymerase. Because of the poor fidelity rate of the HCV polymerase, HCV drug-resistant mutations are likely to occur in patients treated with specific HCV antiviral therapeutics (e.g., protease inhibitors and polymerase inhibitors) as have similarly been observed in patients treated with HIV antiviral therapeutics. In addition to developing resistance to antiviral therapies that specifically target HCV, the virus is capable of developing resistance to non-specific antiviral therapeutics such as interferon.

Drug resistance to protease inhibitors is of particular concern. In vitro studies have shown that mutations in the HCV protease can confer resistance to protease inhibitors. This finding is similar to that of the HIV protease. In HIV, for instance, it has been found that specific mutations in the HIV protease lead to decreased phenotypic susceptibility to HIV protease inhibitors

The invention provides primers, kits and methods which can be used to determine HCV genotype and HCV phenotype. The methods of the invention can be useful, for instance, for predicting the response of a patient to an HCV therapeutic and for determining HCV drug resistance (e.g., protease drug resistance). The methods of the invention can also be useful in preclinical drug development for screening potential protease inhibitors for activity.

### SUMMARY OF THE INVENTION

The present invention is based, in part, on the discovery of primers useful for identifying the sub-type of a hepatitis C virus (HCV) and on the discovery of methods for genotyping and phenotyping HCV. Accordingly, the present invention provides primers, kits comprising the primers, methods for amplifying specific regions of a HCV, e.g., genotype 1 HCV, methods for determining the genotype and phenotype of a HCV, e.g., genotype 1a or 1b, etc. The invention also includes methods for determining the presence of a drug-resistant HCV.

The present invention provides a population of primers. The population of primers can comprise first or second round primers. In one embodiment, each primer comprises a nucleic acid sequence encoding Met/Lys-Glu/Gly-Thr/Ile-Lys-Ile/Val/Leu-Ile/Ala-Thr/Gln-Trp/Lys. In another embodiment, the complement of each primer comprises a nucleic acid sequence encoding Ser-Thr-Tyr-Gly/Cys-Lys-Phe-Leu-Ala-Asp-Gly.

In yet another embodiment, each primer comprises a nucleic acid sequence encoding Ala-Pro/His-Ile-Thr-Ala-Tyr-Ser/Ala-Gln/Arg-Gln-Thr. In still another embodiment, the complement of each primer comprises a nucleic acid sequence encoding Gly-Ser-Gly/Arg-Lys-Ser/Thr-Thr/Asn-Lys/Arg-Val-Pro-Ala/Val-Ala/Asp.

In another embodiment, a kit comprising the primers of the invention is provided.

The invention provides methods of amplifying a nucleic acid encoding a NS3 HCV protease domain. The methods of the invention include, for instance, amplification of a nucleic acid encoding a NS3/4A domain or portion thereof, a NS3 domain or portion thereof (e.g., both NS3 protease and NS3 helicase or NS3 protease and a portion of NS3 helicase), a NS2/NS3 domain or portion thereof, or a NS2/NS3/4A domain or portion thereof.

The invention includes amplifying a HCV protease domain using a first and/or second round of primers. In one embodiment of the invention, the second round of primers (i.e., second set) is nested within the nucleic acid region amplified using the first set of primers. For instance, the invention includes amplifying a HCV nucleic acid encoding a protease domain using a first round of primers comprising nucleic acid sequences encoding Met/Lys-Glu/Gly-Thr/Ile-Lys-Ile/Val/Leu-Ile/Ala-Thr/Gln-Trp/Lys and Ser-Thr-Tyr-Gly/Cys-Lys-Phe-Leu-Ala-Asp-Gly. The second round of primers can comprise nucleic acid sequences encoding Ala-Pro/His-Ile-Thr-Ala-Tyr-Ser/Ala-Gln/Arg-Gln-Thr and Gly-Ser-Gly/Arg-Lys-Ser/Thr-Thr/Asn-Lys/Arg-Val-Pro-Ala/Val-Ala/Asp.

The invention includes methods of amplifying a nucleic acid sample from a sample of a patient infected, or suspected of being infected, with HCV using the primers of the invention. In one aspect of the invention, a HCV protease domain is amplified using genotype non-specific degenerate primers. In another aspect of the invention, a HCV protease domain is amplified using genotype specific non-degenerate primers. In yet another aspect of the invention, a HCV protease domain is amplified using locked nucleic acid primers.

It is possible using the primers of the invention to genotype a HCV based solely on variations in the nucleic acid sequence which encodes the NS3 protease domain. The invention includes genotyping HCV by nucleic acid based methods known in the art (e.g., PCR amplification, sequencing and hybridization methods) using the primers of the invention.

In one embodiment of the invention, a HCV genotype is determined by amplifying and/or sequencing the NS3 protease domain. For instance, the invention includes methods of amplifying a nucleic acid fragment within the protease domain of the HCV and determining the genotype of the HCV based on the sequence of the fragment. In one aspect of the invention, the genotype of a HCV is determined by amplifying a HCV protease domain using genotype non-specific degenerate primers and sequencing the amplified nucleic acid product. In another aspect of the invention, the genotype of a HCV is determined by amplifying a HCV protease domain using genotype specific non-degenerate primers and sequencing the amplified nucleic acid product. For instance, using the methods of the invention, HCV genotype can be determined by comparing a target sequence to sequence(s) of known genotype(s), e.g., 1a and/or 1b HCV.

The invention also provides methods of determining the presence of a drug resistant HCV, e.g., in patients with genotype 1 HCV. In one embodiment, the presence of drug resistant HCV is determined by amplification of a nucleic acid fragment encoding the NS3 protease domain and determining the presence of a mutation associated with drug resistance within the fragment. The presence of the mutation is indicative of the drug resistant HCV. In one embodiment, the amplified nucleic acid encodes a mutation at position D168, for instance, D168A and D168V. The nucleic acid may encode additional mutations that confer drug resistance, including, but not limited to mutations at NS3 positions A156 (e.g., A156T and A156S) and F43 (e.g., F43S). In one example, primers U3420 and D4038 described in Example 2 can be used to identify these drug resistant related mutations.

The invention further provides a method of determining the phenotype of a HCV (e.g., protease activity). The method comprises cloning the NS3 protease domain of HCV into a screening vector comprising a polynucleotide encoding one or more HCV membrane associating proteins and a secretable reporter (e.g., a secretable luciferase reporter). In one embodiment, the screening vector includes maximum number of HCV membrane associating proteins allowed in the vector, e.g., 4A, 4B, and 5A (e.g., to minimize or reduce non-specific background noise signal) and a secretable luciferase reporter. In another embodiment, the screening vector includes HCV NS3 Helicase, 4A, 4B, 5A, 5B (e.g., first 6 amino acids of 5B), and a secretable luciferase reporter. In yet another embodiment, the screening vector includes HCV NS3 Helicase, 4A, 4B (e.g., the first 6 amino acids of 4B) and a secretable luciferase reporter. In still yet another embodiment, the screening vector includes HCV NS3 Helicase, 4A, 4B, 5A (e.g., the first 6 amino acids of 5A) and a secretable luciferase reporter.

The screening vector expresses a polynucleotide encoding the NS3 protease domain, one or more additional HVC NS domains (e.g., NS3 Helicase, 4A, 4B, 5A) and a secretable reporter, wherein the NS3 protease domain, one or more HCV NS domains, and the secretable reporter are operably linked. If the NS3 protease domain is functional, i.e., is capable of cleaving the polyprotein, the reporter (e.g., luciferase) is cleaved and secreted. A signal from the secreted reporter can be detected outside of the cell in the presence of an appropriate substrate (e.g., a luciferase substrate). If the NS3 protease domain is not functional, the secreted reporter is not cleaved and thus not secreted (i.e., is not capable of producing a signal outside of the cell).

In one embodiment of the invention, a HCV is isolated and screened for susceptibility to a HCV antiviral therapeutic such as a protease inhibitor. This method comprises cloning a NS3 protease domain of the isolated HCV into a screening vector described herein (e.g., a screening vector comprising a polynucleotide encoding HCV NS3 Helicase, 4A, 4B, 5A, 5B (e.g. first 6 amino acids of 5B) and a secreted luciferase reporter). Transfected cells are contacted with a candidate therapeutic such as a protease inhibitor. If the HCV is susceptible to the therapeutic, the NS3 protease does not cleave the secreted luciferase reporter and the secreted luciferase reporter is not secreted. Such a method can be useful, for instance, for preclinical screening of compounds for antiviral activity, determining whether a patient will respond to a particular antiviral therapy, and determining whether a patient is resistant to a particular antiviral therapy.

### BRIEF DESCRIPTION OF THE DRAWINGS

FIG. 1 shows a flow chart depicting exemplary steps of a method of determining resistance of HCV obtained from a patient.

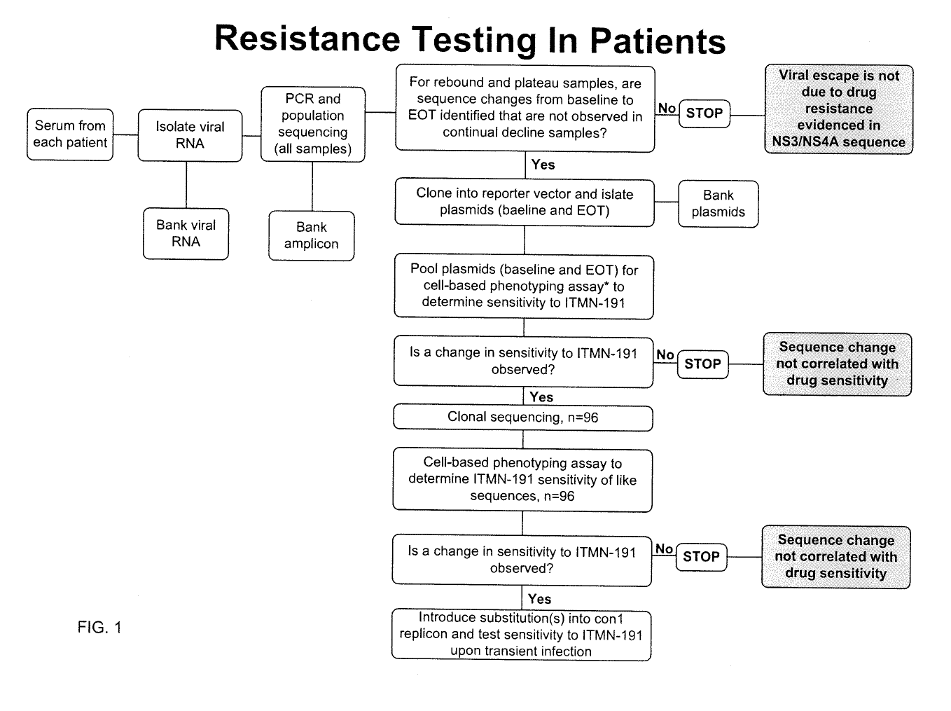

FIG. 2 shows an exemplary reporter system.

FIG. 3 shows phenotyping assay results with DNA from a 96-well Mini-Prep.

FIG. 4 shows the phenotyping assay signal variation across a 96-well plate.

FIG. 5 shows the EC50 variation of the same NS3 sequence using the phenotyping system.

FIG. 6 shows a flow chart depicting exemplary steps of an automation protocol for a cell-based reporter assay.

FIG. 7 shows the genotyping and phenotyping of the HCV NS3 protease domain using exemplary primers of the invention.

FIG. 8 shows the amino acid conservation among genotype 1 isolates in upstream primer, U3276.

FIG. 9 shows the amino acid conservation among genotype 1 isolates in downstream primer, D4221.

FIG. 10 shows the amino acid conservation among genotype 1 isolates in upstream primer, U3420.

FIG. 11 shows the amino acid conservation among genotype 1 isolates in downstream primer, D4038.

FIG. 12 shows the results of a genotyping assay using genotype 1 a/b non-specific degenerate primers.

FIG. 13 shows the results of phenotyping patient NS3 clones.

FIG. 14 shows the sequences of the 1a/b-specific non-degenerate primers.

FIG. 15 shows results obtained with genotype 1a/b-specific non-degenerate primers.

FIG. 16 shows the reproducibility of the phenotyping assay of the invention.

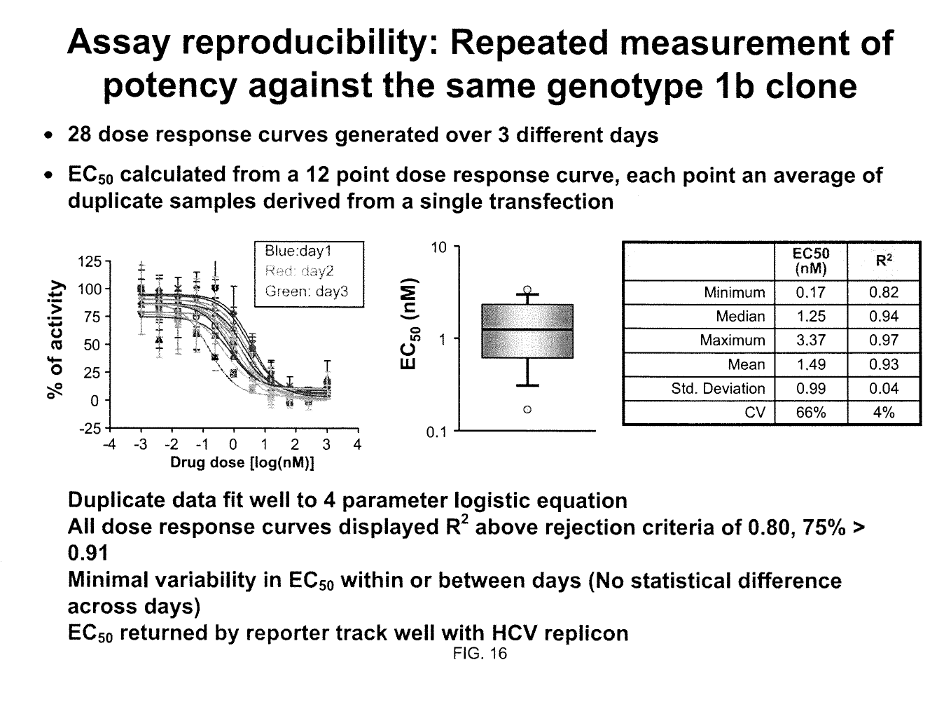

FIG. 17 shows quasi-species analysis with clinical samples.

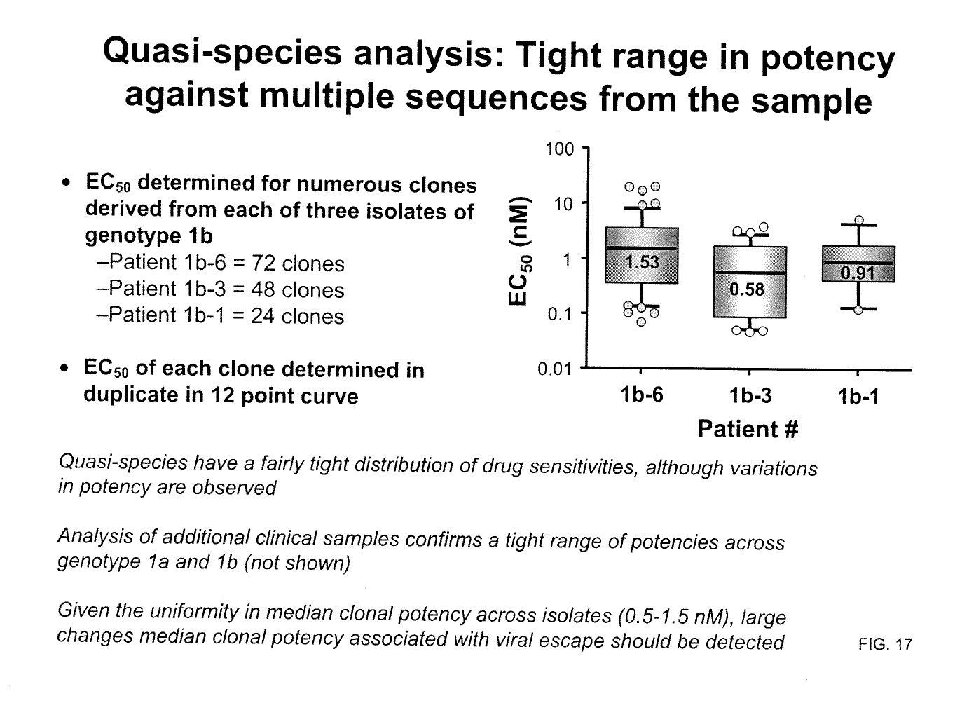

FIG. 18 shows the comparison between population phenotyping and clonal phenotyping.

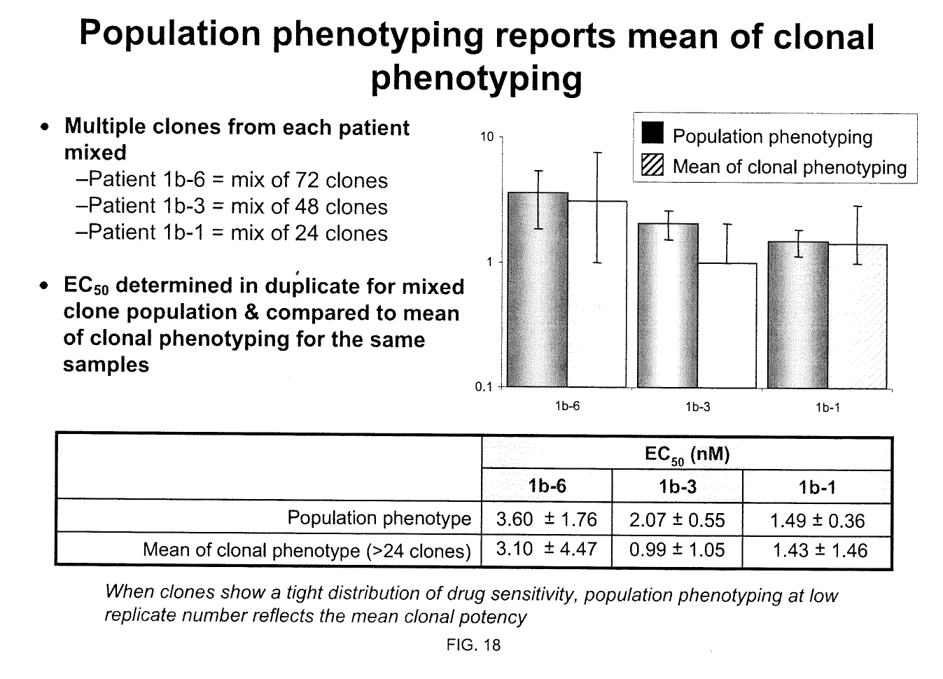

FIG. 19 shows that the phenotyping assay reports potencies against variants identified in in vitro resistance studies.

FIG. 20 shows the characterization of in vitro identified substitutions using the phenotyping assay in a mixed population analysis.

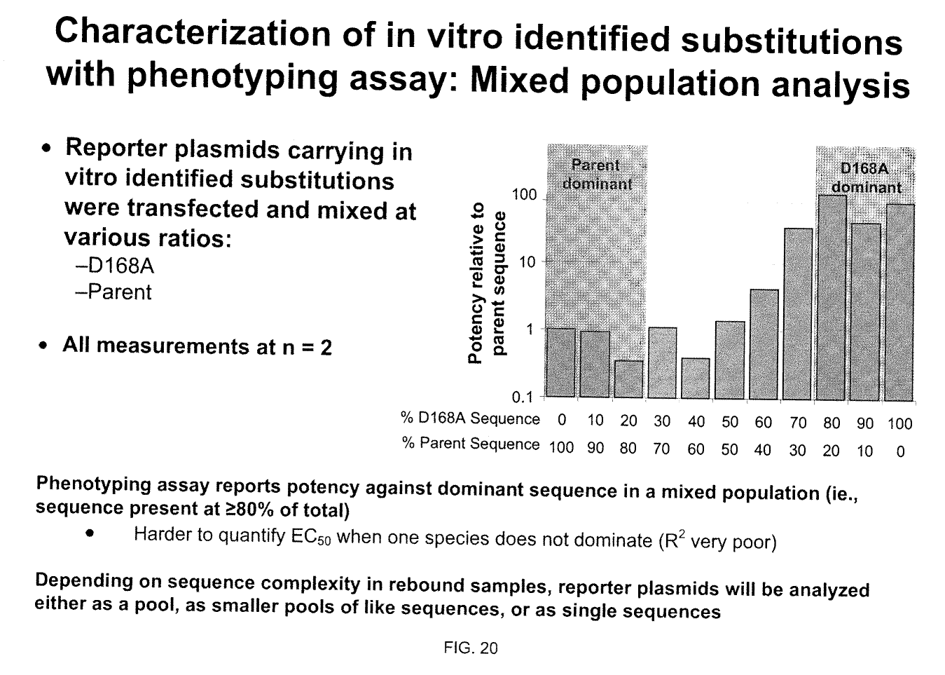

### DETAILED DESCRIPTION

### General Description

The invention includes HCV primers that are capable of annealing to a HCV nucleic acid encoding one or more NS3 domains. The invention also includes methods of amplifying a nucleic acid that encodes one or more NS3 domains, methods of genotyping, e.g. subgenotyping HCV, methods of detecting drug resistant mutations and methods of phenotyping HCV. The compositions and methods of the invention are based, in part, on the finding that genetic variations within viral nucleic acids encoding one or more NS3 domains can be used exclusively (i.e., without knowledge of variations outside of the NS3 protease moiety) to determine the genotype of HCV, HCV susceptibility to antiviral therapy and HCV resistance to an antiviral therapeutic (i.e., drug).

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

### DEFINITIONS

As used herein, the term “EC₅₀” or half maximal effective concentration, refers to the concentration of a drug which induces a response halfway between the baseline and maximum. EC₅₀ represents the plasma concentration of a drug required to obtain 50% of the maximum effect in vivo. The term “IC₅₀” or the half maximal inhibitory concentration, represents the concentration of a drug or inhibitor that is required for 50% inhibition in vitro.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, “genotyping” or a “genotypic assay” is a determination of a genetic sequence of an organism, a part of an organism, a gene or a part of a gene. Such assays can be performed in viruses such as HCV to determine the likelihood that a subject will respond favorably to a particular treatment. Such assays can also be performed to determine whether mutations associated with drug resistance are present.

As used herein, the term “HCV cassette” refers to a HCV NS3 nucleic acid sequence from a patient (i.e., a clinical HCV isolate). The HCV cassette may optionally contain a nucleic acid encoding additional NS domains. For example, the HCV cassette may contain NS3 proteinase domain, all or a portion of NS3 helicase domain and/or all or a portion of NS4A.

As used herein, the term “isolated” refers to viral nucleic acids or proteins of interest that are separated from a clinical specimen (e.g., blood, serum) and/or other viral components (e.g., other proteins or nucleic acids). As used herein, an “isolated” nucleic acid sequence refers to a nucleic acid sequence which is essentially free of other nucleic acid sequences, e.g., at least about 20% pure, preferably at least about 40% pure, more preferably about 60% pure, even more preferably about 80% pure, most preferably about 90% pure, and even most preferably about 95% pure. The purity of isolated nucleic acids can be determined, e.g., by agarose gel electrophoresis. An isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated.

As used herein, the terms “nucleic acid,” “polynucleotide,” “oligonucleotide” and “primer” refer to deoxyribonucleotides or ribonucleotides and polymers thereof in either single- or double-stranded form. Thus, when included in a polynucleotide, the most common naturally occurring encoding nucleotides are abbreviated as follows: adenine (A), guanine (G), cytosine (C), thymine (T) and uracil (U). In addition, the letters R and Y represent the purines (A or G) and pyrimidines (C or T), respectively. The letter K represents G or T; the letter S represents G or C; and the letter V represents A, G or C. Further, the superscript “M” following a nucleotide indicates that the nucleotide is modified, e.g., AM, GM, CM and TM indicate a modified adenine, guanine, cytosine and thymine, respectively. Examples of modified nucleotides include, but are not limited to, a Locked Nucleic Acid (LNA). Unless specified otherwise, single-stranded nucleic acid sequences are represented as a series of one-letter abbreviations in a 5′->3′ direction.

Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Cassol et al. (1992); Rossolini et al. (1994) Mol. Cell. Probes 8:91-98). The term nucleic acid is used interchangeably with gene, cDNA, and mRNA encoded by a gene.

An upstream primer generally binds to a region that is closer to the 5′ end of the nucleic acid molecule as compared to the region on the nucleic acid that is to be amplified. A downstream primer, on the other hand, generally binds to a region that is closer to the 3′ end of the nucleic acid molecule as compared to the region on the nucleic acid that is to be amplified.

As used herein, a DNA segment is referred to as “operably linked” when it is placed into a functional relationship with another DNA segment. For example, DNA for a signal sequence is operably linked to DNA encoding a fusion protein of the invention if it is expressed as a preprotein that participates in the secretion of the fusion protein; a promoter or enhancer is operably linked to a coding sequence if it stimulates the transcription of the sequence. Generally, DNA sequences that are operably linked are contiguous and in reading phase. Similarly, a polypeptide sequence is “operably linked” when it is placed into a functional relationship with another polypeptide sequence. Thus, in certain embodiments, a protease sequence that is operably linked to another peptide sequence is linked such that the protease sequence, if functional, is capable of proteolysing at least one peptide bond in the linked peptide sequence.

As used herein, “phenotyping” or “phenotypic assay” refers to methods of determining phenotypic characteristics of a HCV virus, for instance, susceptibility to an antiviral agent and/or antiviral therapeutic resistance. Such assays can be performed to establish whether certain mutations associated with drug resistance are present in a HCV specimen.

As used herein, the term “polypeptide” refers to a compound made up of a single chain of amino acid residues linked by peptide bonds. The conventional three-letter or single letter codes for amino acid residues are used herein wherein alanine is ala or A; arginine is arg or R; asparagine is asn or N; aspartic acid is asp or D; cysteine is cys or C; glutamic acid is glu or E; glutamine is gln or Q; glycine is gly or G; histidine is his or H; isoleucine is ile or I; leucine is leu or L; lysine is lys or K; methionine is met or M; phenylalanine is phe or F; proline is pro or P; serine is ser or S; threonine is thr or T; tryptophan is trp or W; tyrosine is tyr or Y and valine is val or V.

Unless noted otherwise, when polypeptide sequences are presented as a series of one-letter and/or three-letter abbreviations, the sequences are presented in the N->C direction, in accordance with common practice. A number following the abbreviation indicates the position of that amino acid, from the N-terminal end (e.g., Met-1 represents a methionine at position 1).

As used herein, the term “recombinant” refers to a cell, tissue or organism that has undergone transformation with a new combination of genes or DNA.

As used herein, the term “subject” can be a human, a mammal, or an animal. The subject being treated is a patient in need of treatment or potentially in need of treatment. “Subject” and “patient” are used interchangeably herein.

The term “substantially complementary” in reference to primer is used herein to mean that the primer is sufficiently complementary to hybridize selectively to a nucleotide sequence under the designated annealing conditions, such that the annealed primer can be extended by a polymerase to form a complementary copy of the nucleotide sequence.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell. “Transformation” as used herein includes transfection.

As used herein, the term “transformant” refers to a cell, tissue or organism that has undergone transformation or transfection.

As used herein, the term “vector” refers broadly to any plasmid, phagemid or virus encoding an exogenous nucleic acid. The term is also be construed to include non-plasmid, non-phagemid and non-viral compounds which facilitate the transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant cytomegalovirus, recombinant vaccinia virus, a recombinant adenovirus, a recombinant retrovirus, a recombinant adeno-associated virus, a recombinant avian pox virus, and the like (Cranage et al., 1986, EMBO J. 5:3057-3063; International Patent Application No. WO 94/17810, published Aug. 18, 1994; International Patent Application No. WO 94/23744, published Oct. 27, 1994). Examples of non-viral vectors include, but are not limited to, liposomes, polyamine derivatives of DNA, and the like.

As used herein, the term “wild type” refers to a polynucleotide or polypeptide sequence that is naturally occurring.

HCV Primers

The invention provides a population of primers comprising at least 1, 2, 3, 4 or 5 primers. In one embodiment, the one or more primers are capable of annealing (i.e., hybridizing) to a region of a HCV genome. In one embodiment, the one or more primers are capable of annealing to a nucleic acid encoding a HCV protease. In another embodiment, the one or more primers are capable of annealing to a nucleic acid encoding a NS3 protease domain. In yet another embodiment, the one or more primers are capable of annealing to a nucleic acid encoding a NS3/4A domain or a portion thereof.

In another embodiment of the invention, the one or more primers disclosed herein are capable of amplifying a region of a HCV genome under conditions necessary for nucleic acid amplification. In one instance, the one or more primers are capable of amplifying a nucleic acid encoding a HCV protease. In one instance, the one or more primers are capable of amplifying a nucleic acid encoding a NS3 protease domain, or a portion thereof. In yet another instance, the one or more primers are capable of amplifying a nucleic acid encoding a NS3/4A domain or a portion thereof.

The primers can be genotype specific or degenerate primers. Further, the primers can be upstream primers or downstream primers and could be used during the first round, second round, or any subsequent round of amplification. The primers can be used with any known method of amplification, including, but not limited to, polymerase chain reaction (“PCR”), real-time polymerase chain reaction (“RT-PCR”), ligase chain reaction (“LCR”), self-sustained sequence replication (“3SR”) also known as nucleic acid sequence based amplification (“NASBA”), Q-B-Replicase amplification, rolling circle amplification (“RCA”), transcription mediated amplification (“TMA”), linker-aided DNA amplification (“LADA”), multiple displacement amplification (“MDA”), invader and strand displacement amplification (“SDA”).

In one embodiment, each primer in the population comprises a nucleic acid sequence encoding a polypeptide with an amino acid sequence:

(SEQ ID NO: 1)

Met/Lys-Glu/Gly-Thr/Ile-Lys-Ile/Val/Leu-Ile/Ala-
Thr/Gln-Trp/Lys.

The primer of SEQ ID NO: 1 can be an upstream or a downstream primer and can be used during a first or a subsequent round of amplification. In some embodiments, the primer of SEQ ID NO: 1 is an upstream primer. In other embodiments, the primer is used during the first round of amplification.

The percentages of Met-1, Glu-2, Thr-3, Ile-6, Thr-7, and Trp-8 of the amino acid sequence of SEQ ID NO: 1 range from about 95% to about 99.9%, and the percentages of Lys-1, Gly-2, Ile-3, Ala-6, Gln-7 and Lys-8 correspondingly range from about 5% to about 0.1%. In some embodiments, the percentages of Met-1, Glu-2, Thr-3, Ile-6, Thr-7, or Trp-8 are about 95%, 96%, 97%, 98%, 99%, 99.1%, 99.2%, 99.3%, 99.4%, 99.5%, 99.6%, 99.7%, 99.8% or 99.9%. The percentages of Lys-1, Gly-2, Ile-3, Ala-6, Gln-7 and Lys-8 change accordingly, so that the sum of the two percentages for each amino acid position is 100%.

The percentages of Ile-5, Val-5 and Leu-5 of the amino acid sequence of SEQ ID NO: 1 range from about 67% to about 72%, from about 14% to about 18% and from about 12% to about 17%, respectively. In some embodiments, the percentage of Ile-5 is about 67%, 68%, 69%, 69.1%, 69.2%, 69.3%, 69.4%, 69.5%, 70%, 71% or 72%; the percentage of Val-5 is about 14%, 15%, 15.9%, 16%, 16.1%, 16.2%, 16.3%, 17% or 18%; and the percentage of Leu-5 is about 12%, 13%, 14%, 14.4%, 14.5%, 14.6%, 14.7%, 14.8%, 15%, 16% or 17%, wherein the sum of the percentages for each amino acid is 100%.

In an exemplary embodiment, the population of primers comprise nucleic acid sequences that encode a population of amino acid sequences that has the following distribution with respect to each amino acid: Met (99.5%)/Lys (0.5%)-Glu (99.5%)/Gly (0.5%)-Thr (96.5%)/Ile (3.5%)-Lys (100%)-Ile (69.3%)/Val (16.1%)/Leu (14.6%)-Ile (99.5%)/Ala (0.5%)-Thr (99.5%)/Gln (0.5%)-Trp (99.5%)/Lys (0.5%)

In another exemplary embodiment, each primer comprises a nucleic acid sequence that is 80%, 85%, 90%, 95%, 96%, 97%, 98%, 99% or 100% homologous to the nucleic acid sequence: ATGGAGACYAAGVTYATYACSTGGG (SEQ ID NO: 2), wherein any of the nucleotides could be modified. Examples of modified nucleotides include, but are not limited to, Locked Nucleic Acid (LNA). Although the primers of the invention can comprise any number of modified bases, e.g., primers with LNA, in some embodiments, the primers comprise about 4 or about 8 LNA. In general, modifications at As and Ts are more common than those at Gs and Cs. In some embodiments, each primer comprises the nucleic acid sequence: ATGGAGACYAMAMGVTYAMTYAMCSTGGG, or AMTGMGMAGACYAMAMGVTYAMTYAMCMSTGGG.

In another embodiment, the complement of each primer in the population comprises a nucleic acid sequence encoding a polypeptide with an amino acid sequence:

(SEQ ID NO: 3)

Ser-Thr-Tyr-Gly/Cys-Lys-Phe-Leu-Ala-Asp-Gly..

The primer of SEQ ID NO: 3 can be an upstream or a downstream primer and can be used during a first or a subsequent round of amplification. In some embodiments, the primer of SEQ ID NO: 3 is a downstream primer. In other embodiments, the primer is used during the first round of amplification. The percentage of Gly-4 of the amino acid sequence of SEQ ID NO: 3 ranges from about 95% to about 99%, and the percentage of Cys-4 correspondingly ranges from about 5% to about 1%. In some embodiments, the percentage of Gly-4 is about 95%, 96%, 97%, 98%, or 99%. The percentage of Cys-4 is correspondingly 5%, 4%, 3%, 2% or 1%.

In an exemplary embodiment, the complements of primers in the population comprise nucleic acid sequences that encode a population of amino acid sequences that has the following distribution with respect to each amino acid: Ser (100%)-Thr (100%)-Tyr (100%)-Gly (97.0%)/Cys (3.0%)-Lys (100%)-Phe (100%)-Leu (100%)-Ala (100%)-Asp (100%)-Gly (100%).

In another exemplary embodiment, each primer comprises a nucleic acid sequence that is 80%, 85%, 90%, 95%, 96%, 97%, 98%, 99% or 100% homologous to the nucleic acid sequence: CCGTCGGCAAGRAACTTGCCRTAGGTGGA (SEQ ID NO: 4), wherein any of the nucleotides could be modified. Examples of modified nucleotides include, but are not limited to, LNA. In some embodiments, each primer comprises the nucleic acid sequence:

CCGTCGGCAAGRAMACTTMGCCRTMAGGTMGGA,
or
CCGTMCGGCAAMGRAMACTMTMGCCRTMAMGGTMGGA.

In yet another embodiment, each primer in the population comprises a nucleic acid sequence encoding a polypeptide with an amino acid sequence:

(SEQ ID NO: 5)

Ala-Pro/His-Ile-Thr-Ala-Tyr-Ser/Ala-Gln/Arg-Gln-
Thr.

The primer of SEQ ID NO: 5 can be an upstream or a downstream primer and can be used during a first or a subsequent round of amplification. In some embodiments, the primer of SEQ ID NO: 5 is an upstream primer. In other embodiments, the primer is used during the second round of amplification.

The percentages of Pro-2, and Gln-8 of the amino acid sequence of SEQ ID NO: 5 range from about 95% to about 99.9%, and the percentages of His-2, and Arg-8 correspondingly range from about 5% to about 0.1%. In some embodiments, the percentages of Pro-2 or Gln-8 are about 95%, 96%, 97%, 98%, 99%, 99.1%, 99.2%, 99.3%, 99.4%, 99.5%, 99.6%, 99.7%, 99.8% or 99.9%. The percentages of His-2 and Arg-8 change accordingly, so that the sum of the two percentages for each amino acid position is 100%.

The percentages of Ser-7 and Ala-7 of the amino acid sequence of SEQ ID NO: 5 range from about 64% to about 68%, and from about 32% to about 36%, respectively. In some embodiments, the percentage of Ser-7 is about 64%, 65%, 66%, 67%, or 68%; and the percentage of Ala-7 is about 32%, 33%, 34%, 35% or 36%, wherein the sum of the percentages for each amino acid is 100%.

In an exemplary embodiment, the population of primers comprise nucleic acid sequences that encode a population of amino acid sequences that has the following distribution with respect to each amino acid: Ala (100%)-Pro (99.5%)/His (0.5%)-Ile (100%)-Thr (100%)-Ala (100%)-Tyr (100%)-Ser (66%)/Ala (34%)-Gln (99%)/Arg (1%)-Gln (100%)-Thr (100%).

In another exemplary embodiment, each primer comprises a nucleic acid sequence that is 80%, 85%, 90%, 95%, 96%, 97%, 98%, 99% or 100% homologous to the nucleic acid sequence: GCGCCYATYACGGCCTAYKCCCARCARAC (SEQ ID NO: 6), wherein any of the nucleotides could be modified. Examples of modified nucleotides include, but are not limited to, LNA. In some embodiments, each primer comprises the nucleic acid sequence:

GCGCCYAMTMYACGGCMCTMAMYKCCCMARCMAMRAC,
or
GCGCCYAMTMYACGGCCTAMYKCCCARCAMRAC.

In yet another exemplary embodiment, each primer comprises a nucleic acid sequence that is 80%, 85%, 90%, 95%, 96%, 97%, 98%, 99% or 100% homologous to the nucleic acid sequence: AGGGCATTTAAATAGCCACCATGGCGCCYATYACGGCCTAYKCCCARCARAC (SEQ ID NO: 7), or AAAAAGGCGCGCCACCATGGCGCCYATYACGGCCTAYKCCCARCARAC (SEQ ID NO: 8), wherein any of the nucleotides of the nucleic acid sequences SEQ ID NO: 7 or 8 could be modified.

Exemplary primers further include those that comprises the nucleic acid sequence:

AGGGCATTTAAATAGCCACCATGGCGCCYAMTMYACGGCCTAMYKCCCAR
CAMRAC,
or
AGGGCATTTAAATAGCCACCATGGCGCCYAMTMYACGGCMCTMAMYKCCC
MARCMAMRAC.

In still another embodiment, the complement of each primer in the population comprises a nucleic acid sequence encoding a polypeptide with an amino acid sequence: Gly-Ser-Gly/Arg-Lys-Ser/Thr-Thr/Asn-Lys/Arg-Val-Pro-Ala/Val-Ala/Asp (SEQ ID NO: 9). The primer of SEQ ID NO: 9 can be an upstream or a downstream primer and can be used during a first or a subsequent round of amplification. In some embodiments, the primer of SEQ ID NO: 9 is a downstream primer. In other embodiments, the primer is used during the second round of amplification.

The percentages of Gly-3, Ser-5, Thr-6, Ala-10, and Ala-11 of the amino acid sequence of SEQ ID NO: 9 range from about 95% to about 99.9%, and the percentages of Arg-3, Thr-5, Asn-6, Val-10, and Asp-11 correspondingly range from about 5% to about 0.1%. In some embodiments, the percentages of Gly-3, Ser-5, Thr-6, Ala-10 or Ala-11 are about 95%, 96%, 97%, 98%, 99%, 99.1%, 99.2%, 99.3%, 99.4%, 99.5%, 99.6%, 99.7%, 99.8% or 99.9%. The percentages of Arg-3, Thr-5, Asn-6, Val-10 and Asp-11 change accordingly, so that the sum of the two percentages for each amino acid position is 100%.

The percentages of Lys-7 and Arg-7 of the amino acid sequence of SEQ ID NO: 9 range from about 90% to about 95% and from about 10% to about 5%, respectively. In some embodiments, the percentage of Lys-7 is about 90%, 91%, 92%, 93%, 94% or 95%; and the percentage of Arg-7 is correspondingly about 10%, 9%, 8%, 7%, 6% or 5%.

In an exemplary embodiment, the complements of primers in the population comprise nucleic acid sequences that encode a population of amino acid sequences that has the following distribution with respect to each amino acid: Gly (100%)-Ser (100%)-Gly (99.5%)/Arg (0.5%)-Lys (100%)-Ser (99.5%)/Thr (0.5%)-Thr (99%)/Asn (1%)-Lys (93%)/Arg (7%)-Val (100%)-Pro (100%)-Ala (99%)/Val (1%)-Ala (99%)/Asp (1%).

In another exemplary embodiment, each primer comprises a nucleic acid sequence that is 80%, 85%, 90%, 95%, 96%, 97%, 98%, 99% or 100% homologous to the nucleic acid sequence: GCAGCCGGCACYTTRGTGCTYTTRCCGCTRCC (SEQ ID NO: 10), wherein any of the nucleotides could be modified. Examples of modified nucleotides include, but are not limited to, LNA. In some embodiments, each primer comprises the nucleic acid sequence: GCAGCCGGCAMCYTTMRGTMGCTMYTMTMRCMCGCTMRCC, or GCAGCCGGCACYTTMRGTGCTMYTTMRCCGCTMRCC. In yet another exemplary embodiment, each primer comprises a nucleic acid sequence that is 80%, 85%, 90%, 95%, 96%, 97%, 98%, 99% or 100% homologous to the nucleic acid sequence: AAAAAGCGGCCGCAGCCGGCACYTTRGTGCTYTTRCCGCTRCC, (SEQ ID NO: 11), or CTTGGTTAATTAATGCAGCCGGCACYTTRGTGCTYTTRCCGCTRCC (SEQ ID NO: 12), wherein any of the nucleotides of the nucleic acid sequences SEQ ID NO: 11 or 12 could be modified.

Exemplary primers further include those that comprises the nucleic acid sequence:

AAAAAGCGGCCGCAGCCGGCACYTTMRGTGCTMYTTMRCCGCTMRCC,
or
AAAAAGCGGCCGCAGCCGGCAMCYTTMRGTMGCTMYTMTMRCMCGCTMRC
C.

In another exemplary embodiment, non-degenerate primers or primers specific for certain genotype or subgenotype are provided. Exemplary primers for HCV genotype 1a includes ATGGAGACCAAGCTCATCACGTGGG (e.g., upstream primer), ACCCGCCGTCGGCAAGGAACTTGCCGTA (e.g., downstream primer), AGGGCATTTAAATAGCCACCATGGCGCCCATCACGGCGTACGCCCAGCAGAC (e.g., upstream primer), AAAAAGCGGCCGCAGCCGGGACCTTGGTGCTCTTACCGCTGCC (e.g., downstream primer). Exemplary primers for HCV genotype 1b includes ATGGAGACCAAGATCATCACCTGGG (e.g., upstream primer), CCGTCGGCAAGGAACTTGCCATAGGTGGA (e.g., downstream primer), AGGGCATTTAAATAGCCACCATGGCGCCCATCACGGCCTACTCCCAACAGAC (e.g., upstream primer), AAAAAGCGGCCGCAGCCGGCACCTTAGTGCTCTTGCCGCTGCC (e.g., downstream primer).

Kits

The invention includes a kit comprising one or more primers of the invention or complements thereof. The kit can comprise any combination of primers of the invention. In one embodiment, the kit comprises the one or more primers encoding the polypeptide sequences of SEQ ID NO: 1 and/or SEQ ID NO: 3 or complements thereof. In another embodiment, the kit comprises one or more primers encoding the polypeptide sequences of SEQ ID NO: 5 and/or SEQ ID NO: 9 or complements thereof. In still another embodiment, the kit comprises one or more primers of SEQ ID NO: 2 and/or SEQ ID NO: 4 or complements thereof. In yet another embodiment, the kit comprises one or more primers of SEQ ID NOs: 6, 7 and/or 8 or complements thereof. In yet another embodiment, the kit comprises one or more primers of SEQ ID NOs: 10, 11 and/or 12 or complements thereof. In yet another embodiment, the kit comprises one or more primers of SEQ ID NOs: 6, 7 and/or 8 or complements thereof and one or more primers of SEQ ID NOs: 10, 11 and/or 12 or complements thereof.

In yet another embodiment, the kit further comprises instructions for amplifying a region of a HCV, determining the genotype or phenotype of a HCV, or determining the presence of a drug resistant HCV. For instance, a kit can comprise instructions for determining the susceptibility of HCV to a therapeutic. Such kits can be used to screen candidate therapeutics for antiviral effects.

In yet another embodiment, the kit further comprises one or more enzymes or reagents for amplifying a HCV nucleic acid. For instance, in one embodiment, the kit comprises one or more primers and a polymerase (e.g., thermophilic polymerase such as Taq polymerase or a mesophilic polymerase). In yet another embodiment, the kit comprises dNTPs. The kit may optionally contain amplification buffer.

The kit of the invention may comprise one or more primers in a labeled container or multiple labeled containers.

In another embodiment of the invention, the kit comprises a vector for cloning a HCV nucleic acid. For instance, the invention includes a kit comprising a vector with a CMV promoter. The invention also includes a kit comprising a vector encoding a reporter moiety, for instance, a luciferase moiety. In one embodiment of the invention, the kit includes a luciferase substrate such as luciferin. In yet another embodiment of the invention, the kit comprises one or more primers and a vector for cloning a HCV nucleic acid.

Amplification of HCV

The invention includes methods of amplifying a HCV nucleic acid. The methods of the present invention can be used to amplify DNA or RNA. The molecule may be in either a double-stranded or single-stranded form, preferably, double-stranded. Where the nucleic acid as starting material is double-stranded, it is preferred to render the two strands into a single-stranded, or partially single-stranded, form. Methods known to separate strands includes, but not limited to, heating, alkali, formamide, urea and glycoxal treatment, enzymatic methods (e.g., helicase action) and binding proteins. For instance, the strand separation can be achieved by heating at temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

In one embodiment, the method comprises amplifying a nucleic acid from an HCV sample using the primers of the invention. The HCV sample, may be, for example, a clinical specimen from a patient infected, or suspected of being infected, with HCV. Clinical specimens containing HCV can be obtained, for instance, by venipuncture or biopsy (e.g., blood and liver samples). Viral nucleic acids can be amplified directly from the specimen (i.e., no isolation and/or purification steps prior to amplification) or can be isolated and purified by methods known in the art prior to amplification.

The methods of the invention can be used to amplify a protease region of HCV. For example, the invention includes methods of amplifying a NS3 protease domain of a HCV. One or more of the primers of the invention, e.g., those of SEQ ID NO: 1-12, can be used to amplify a region of a HCV. The primers may contain one or more modified nucleotides. In an exemplary embodiment, primers of SEQ ID NO: 1 and SEQ ID NO: 3, or those of SEQ ID NO: 5 and SEQ ID NO: 9, or combinations thereof are used. In another exemplary embodiment, the primers of SEQ ID NO: 2 and SEQ ID NO: 4, or those of SEQ ID NOs: 6, 7 or 8 and SEQ ID NOs: 10, 11 or 12, or combinations thereof are used to amplify viral nucleic acids.

The invention includes methods of amplifying nucleic acids encoding NS3 using full-length NS3/4A primers. In one embodiment, primers are full-length NS3/4A primers specific to a patient infected, or suspected of being infected, with HCV. In another embodiment, primers anneal to a nucleic acid encoding NS3/4A or a portion thereof, NS3 (NS3 protease and NS3 helicase) or a portion thereof, NS2/NS3 or a portion thereof, or NS2/NS3/NS4A or a portion thereof. In another embodiment, the primers encode a HCV protease domain.

One or more primers can be specific to particular HCV sequence, for instance, genotype specific. In one embodiment of the invention, one or more primers are degenerate primers.

Different primers can be used either simultaneously or sequentially to amplify the desired regions of the HCV. In one embodiment of the invention, two or more sets of primers are used. In this embodiment, a second set of primers can be used to amplify a region within a amplified product of a first set of primers (i.e., at least one set of primers is nested).

Nucleic acids can be amplified with the primers of the invention by methods known in the art such as by polymerase chain reaction (PCR). In PCR, a set of primers of the invention is annealed to single stranded HCV nucleic acid template in the presence of a polymerase and dNTPs under conditions which allow for subsequent elongation of the primers and denaturation. See for instance, U.S. Pat. Nos. 4,683,202 and 5,766,889, each of which is herein incorporated by reference in its entirety. As can be appreciated by a skilled artisan, various amplification methods can be used to amplify a nucleic acid encoding a NS3 protease domain using the primers of the invention by methods known in the art, including non-polymerase chain reaction methods and methods which do not rely on the use of thermophilic polymerases (e.g., mesophilic polymerases such as phi29). Amplification can be carried out using any amplification method now known, or later discovered, including, but not limited to, those described herein.

Methods of Genotyping HCV

The invention further provides methods of determining the genotype of a HCV. Specifically, the present invention allows the determination of a HCV genotype without sequencing the entire HCV genome. Rather, the present invention allows one to determine a HCV genotype based solely on the sequence of the HCV protease (e.g., NS3, NS3/4A or NS3 and a portion of NS4A).

In one embodiment, the method comprises amplifying a nucleic acid encoding a protease domain as previously described (e.g., using genotype specific primers or degenerate primers) and determining the genotype of the HCV based on the sequence of said protease encoding nucleic acid fragment. In one embodiment, the fragment is within a NS3 protease domain of the HCV.

The invention also provides methods of determining the genotype of HCV by non-amplification means, for instance, by sequencing or hybridization technology. In one embodiment, a genotype of a HCV is determined by determining whether a NS3 genotype specific primer or set of primers is capable of hybridizing to a HCV nucleic acid.

The genotyping methods of the invention can be used to determine whether a HCV isolate is susceptible to a HCV antiviral therapeutic. For instance, the genotyping methods of the invention can be used to determine whether a HCV isolate NS3 sequence is associated with resistance to an antiviral therapeutic. Without wishing to be bound by a particular theory, the inventors of the invention have found that single amino acid substitutions in the NS3 protease region can confer resistance to antiviral therapeutics whereas amino acid substitutions in the helicase domain and NS4A do not appear to affect resistance, e.g., to protease inhibitors.

In yet another embodiment of the invention, the clinical genotype and subtype of an HCV isolate can be determined. There are 11 major clinical genotypes of HCV, which are further characterized by subtype (designated a, b, c, etc.). It has been shown that certain clinical genotypes are more treatable with current therapies than other genotypes. For instance, genotype 1 is a weak responder to interferon alone compared to genotypes 2 and 3. Accordingly, the methods of the invention can be used to assist a physicians in selecting the most suitable therapeutic (i.e., most effective and safe) for a patient infected with HCV by determining the genotype or subtype of a HCV, e.g. HCV genotype 1 including HCV genotype 1a and 1b.

In order to genotype HCV from a patient, a clinical specimen containing the virus must be obtained from the patient. In one embodiment, the specimen is obtained by biopsy, for instance, liver biopsy. In another embodiment, the specimen is blood.

Viral RNA is isolated from the clinical specimen and used as template for amplification. In one embodiment, the nucleic acid template is amplified using genotype specific primers that are capable of annealing to viral nucleic acid encoding a protease, for instance, NS3 or NS3/4A. In another embodiment, the nucleic acid template is amplified using degenerate primers capable of amplifying viral nucleic acid encoding a protease, for instance, NS3 or NS3/4A. The invention includes methods of genotyping using primers encoding the amino acid sequences of SEQ ID NO: 1 and SEQ ID NO: 3 (or their complements) and/or SEQ ID NO: 5 and SEQ ID NO: 9 (or their complements). The invention also includes methods of genotyping using one or more primers corresponding to SEQ ID NOs: 2, 4, 6, 7, 8, 10, 11 or 12 or complements thereof. In one embodiment of the invention, multiple primer sets are used to amplify NS3 nucleic acids in order to generate multiple amplicons for genotype analysis.

Amplified protease nucleic acids are subsequently sequenced using methods known in the art, for instance, Sanger sequencing. In certain embodiments, the amplified protease nucleic acids are sequenced as a population (e.g., to determine the sequence of NS3 protease for a population of HCV isolated from a subject). In other embodiments, the amplified protease nucleic acids are sequenced individually (e.g., to determine the sequence of NS3 protease for individual HCV isolated from a subject). In one embodiment, amplified protease nucleic acids are cloned prior to sequencing by methods known in the art, including, but not limited to, shotgun sequencing. In other embodiments, the amplified protease nucleic acids are sequenced without first being cloned.

In one embodiment, the genotype of a sequenced protease nucleic acid can be determined by aligning the sequence with one or more protease sequences of known genotype and identifying the protease sequence of known genotype that is homologous to the sequenced protease nucleic acid. The analysis can be performed using computer software (e.g., Blast) and databases on computer readable media known in the art.

In another embodiment, the genotype of a sequenced protease nucleic acid can be determined by comparing key nucleotides (or the amino acids coded for by the sequenced nucleotides) of the sequenced protease nucleic acid to controls of known genotype.

In a preferred embodiment, viral RNA is isolated from a serum sample. The sample is amplified using degenerate primers designed to amplify a NS3 protease regardless of genotype. Amplified nucleic acids are subsequently sequenced.

As can be appreciated by a skilled artisan, the scope of the present invention includes variations of the genotyping methods described above. For instance, HCV can be amplified without first isolating RNA from a viral specimen. Also, primers can be varied so long as the primers are capable of annealing to a viral nucleic acid encoding a protease region. For instance, the amplified region may additionally include nucleic acids encoding surround protein domains such as p7, NS2, NS4A, and/or NS4B, or a portion thereof in addition to NS3. Further, amplified nucleic acids may be cloned prior to sequencing.

Methods of Phenotyping HCV

The invention further provides methods of determining the phenotype of HCV. In one embodiment, the phenotype of interest is protease function (e.g., the level of NS3 protease activity). In another embodiment, the phenotype of interest is susceptibility of HCV to an antiviral therapeutic. For instance, the invention includes methods of determining the susceptibility of HCV to a protease inhibitor. In another embodiment of the invention, the phenotype of interest is resistance to an antiviral therapeutic.

In order to phenotype a HCV from a patient, a clinical sample of HCV is obtained from the subject as previously described. For instance, HCV may be obtained from the patient's serum. A protease domain of HCV is amplified so that the amplicon (i.e., HCV cassette) can be cloned into a screening vector. The screening vector containing the patient HCV cassette can be purified by methods known in the art (e.g., maxi-prep) prior to transfection of host cells.

The phenotyping methods of the invention require a patient HCV cassette. As discussed above, HCV nucleic acid is obtained from the patient and amplified. Nucleic acid encoding the NS3 protease domain can be amplified by the methods disclosed throughout this application. For instance, HCV nucleic acid can be amplified using genotype specific primers. In another embodiment, the HCV nucleic acid is amplified using degenerate primers. The invention includes methods of phenotyping using primers encoding the amino acid sequences of SEQ ID NO: 1 and SEQ ID NO: 3 (or their complements) and/or SEQ ID NO: 5 and SEQ ID NO: 9 (or their complements). The invention also includes methods of phenotyping using one or more primers corresponding to SEQ ID NOs: 2, 4, 6, 7, 8, 10, 11 or 12 or complements thereof.

The phenotyping reporter system of the invention relies on a screening vector that comprises a nucleic acid encoding a reporter moiety, such as a secretable reporter moiety. In one embodiment, the screening vector comprises a polynucleotide encoding one or more HCV membrane associating proteins and a secretable reporter. In another embodiment, the screening vector includes maximum number of HCV membrane associating proteins allowed in the vector, e.g. 4A, 4B, and 5A (e.g., to minimize or reduce non-specific background noise signal) and a secretable reporter. In another embodiment, the screening vector includes HCV NS3 Helicase, 4A, 4B, 5A, 5B (e.g., first 6 amino acids of 5B), and a secretable reporter. In yet another embodiment, the screening vector includes HCV NS3 Helicase, 4A, 4B (e.g., the first 6 amino acids of 4B) and a secretable reporter. In still yet another embodiment, the screening vector includes HCV NS3 Helicase, 4A, 4B, 5A (e.g., the first 6 amino acids of 5A) and a secretable reporter.

In one embodiment, the screening vector comprises a polynucleotide encoding HCV NS3 Helicase, 4A, 4B, 5A, 5B (e.g. the first 6 amino acids of 5B) and a secreted reporter. In this embodiment, a NS3 domain cassette is cloned into the screening vector. The NS3 domain cassette can comprise, for instance, a nucleic acid encoding the NS3 protease domain or the NS3 protease domain and a portion of the helicase domain. In one embodiment of the invention, the cassette comprises a nucleic acid encoding the NS3 protease domain and a portion of the helicase domain, for instance, about 4 amino acids, about 5 amino acids, about 6 amino acids, about 7 amino acids, about 8 amino acids, about 9 amino acids, or about 10 amino acids or more of the helicase domain.

In one embodiment, the screening vector comprises a polynucleotide encoding HCV NS4B, 5A, 5B (e.g. the first 6 amino acids of 5B) and a secreted reporter. In this embodiment, a NS3/4A domain cassette is cloned into the screening vector. In another embodiment of the invention, the cassette comprises a nucleic acid encoding the NS3 domain (i.e., NS3 protease and helicase domains) and a portion of the NS4A domain, for instance, about 4 amino acids, about 5 amino acids, about 6 amino acids, about 7 amino acids, about 8 amino acids, about 9 amino acids, or about 10 amino acids or more of the NS4A domain.

As previously discussed, the HCV cassette is cloned into a screening vector comprising a polynucleotide encoding a reporter (e.g., a secretable reporter). The HCV cassette encoding the NS3 protease domain or full length protease (e.g., NS3 Pro or NS3/4A), the vector nucleic acid encoding NS moieties (e.g., NS4B and 5B), and the vector nucleic acid encoding the secreted reporter are operably linked so that detection of a signal from the secreted reporter indicates that the presence of a functional desired domain. In one embodiment of the invention, the vector is under the control of a CMV promoter.

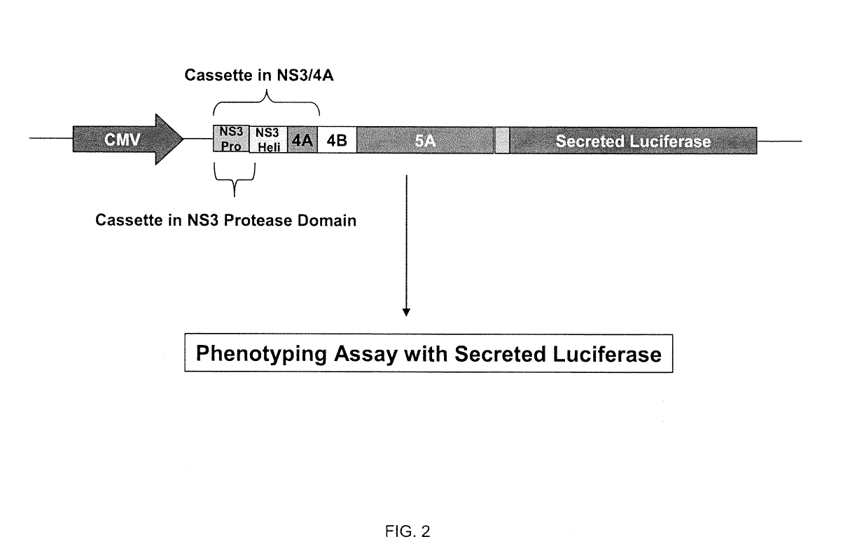

The screening vector of the invention can be transiently transfected in numerous cell lines. In one embodiment, the screening vector is transfected in a 293 cell line (e.g., 293-FS cells). It is important to note that the system is not limited to Huh-7 cells or “cured” replicon cells. An exemplary HCV reporter system that can be used in a phenotyping assay is shown in FIG. 2. As depicted in FIG. 2, NS3 activity is linked with reporter activity (e.g., secreted luciferase activity). The system of FIG. 2 provides easy and consistent transfection of DNA and avoids the problems associated with transfecting RNA.

The reporter system of the invention works by secreting a reporter moiety capable of detection if the NS3 cassette encodes a functional protease. Specifically, a functional protease domain within the polypeptide cleaves the reporter from the translated polypeptide. If the protease domain is not functional, the reporter moiety is not cleaved. Accordingly, detection of a secreted signal indicates the presence of a functional protease domain whereas the absence of a secreted signal indicates the absence of a functional protease domain. In certain embodiments, a weak signal is indicative of an inefficient protease.

The reporter capable of secretion can be any reporter moiety known in the art. In one embodiment, the reporter capable of secretion is a detectable moiety either secretable on its own or secretable after being operably linked to a secretion signal peptide. In another embodiment, the reporter capable of secretion is secretable luciferase. In order for luciferase to provide a detectable signal, a luciferase substrate must be available. In one embodiment of the invention, the host cell containing the vector of the invention is contacted with a luciferase substrate. In one embodiment, a reporter substrate such as luciferin is added to cells and a resulting signal is read. In one embodiment, the signal is a fluorescent signal. In one embodiment, the signal is read by methods known in the art, for instance, using a luminometer. As will be understood by persons skilled in the art, additional secretable reporter moieties can also be used in the system, providing that the reporter moiety is not otherwise present in the cells used to perform the assay. Other suitable reporter moieties include, but are not limited to, SEAP.

In one embodiment, the phenotype assay of the invention can be used to determine whether a HCV isolate is susceptible to an antiviral treatment (e.g., protease inhibitor therapeutic). In this embodiment host cells comprising the screening vector are contacted with a drug. The drug can be serially diluted. If the drug prevents or reduces the secretion of the reporter, the drug is effective at inhibiting the viral protease. To determine whether the drug prevents or reduces the secretion of the reporter, a reporter substrate can be added, for instance, a luciferase substrate can be added if the reporter is luciferase. Signal intensity can then be determined.

In another embodiment, the phenotype assay of the invention can be used to determine whether a population of HCV (e.g., an population of HCV isolated from a subject) is susceptible to an antiviral treatment (e.g., protease inhibitor therapeutic). In this embodiment host cells comprising screening vectors, wherein individual screening vectors can contain nucleic acids encoding different NS3 protease domains from a population of HCV, are contacted with a drug. The drug can be serially diluted, reporter substrate can be added, and reporter activity can be determined, as described above and elsewhere herein.

In another embodiment, drug resistance to a drug of interest is determined by contacting host cells transiently transfected with the screening vector containing the patient's HCV cassette with a drug of interest. In one embodiment, the drug of interest is a protease inhibitor. For instance, the drug of interest may be ITMN-191. In one embodiment of the invention, the drug of interest is serially diluted (e.g., serially diluted ITMN-191). Drug resistance is indicated by the secretion of the reporter. In one embodiment, secretable luciferase is the reporter, and a luciferase substrate is added. After addition of the secretable luciferase substrate, a signal (i.e., fluorescence) is read. Based on the presence and intensity of the signal, an analysis can be performed to determine drug resistance. In one embodiment, the EC50 values can be determined by plotting reporter signals against drug concentrations. Any increase in EC50 value in the phenotyping assay is indicative of drug resistance in patient's HCV.

The phenotyping methods of the invention can be performed in a high-throughput format. The secreted reporter is easy to measure and phenotyping system is amendable to robotics. In one embodiment, automation allows a 12-point EC₅₀ determination for approximately 96, 144, 192, 240 or 288 samples per day.

Nucleic Acids

The invention also provides isolated nucleic acid molecules encoding a NS3 protease domain of a HCV NS polyprotein. For instance, the invention includes isolated nucleic acids molecules encoding a NS3 protease domain and optionally a NS2 domain, NS3 helicase domain, NS4A domain, NS4B domain and/or NS5A domain, or a portion of any such domain. In one embodiment, the isolated nucleic acid molecule encodes a portion of NS2, starting about at amino acid 170. In another embodiment, the nucleic acid molecule encodes a NS3 protease domain and at least a portion of the NS3 helicase domain of the NS polyprotein. For instance, the invention includes an isolated nucleic acid molecule encoding a NS3 protease domain and at least about 4 amino acids, about 5 amino acids, about 6 amino acids, about 7 amino acids, about 8 amino acids, about 9 amino acids, or about 10 amino acids or more of the NS3 helicase domain. In one embodiment of the invention, the isolated nucleic acid molecule encodes a NS3 protease domain, a NS3 helicase domain, and a NS4A domain. For instance, the invention includes an isolated nucleic acid molecule encoding a NS3 protease domain, a NS3 helicase domain, and at least about 4 amino acids, about 5 amino acids, about 6 amino acids, about 7 amino acids, about 8 amino acids, about 9 amino acids, or about 10 amino acids or more of the NS4A domain.

In one embodiment, isolated nucleic acid molecules encoding a NS3 protease domain are obtained using the primers of the invention. For instance, isolated nucleic acid molecules can be obtained using NS3 genotype specific primers. In another embodiment, isolated nucleic acid molecules are obtained using degenerate primers designed to amplify a NS3 region regardless of genotype. The invention includes isolated nucleic acids obtained using primers encoding the amino acid sequences of SEQ ID NO: 1 and SEQ ID NO: 3 (or their complements) and/or SEQ ID NO: 5 and SEQ ID NO: 9 (or their complements). The invention also includes isolated nucleic acids obtained using one or more primers corresponding to SEQ ID NOs: 2, 4, 6, 7, 8, 10, 11 or 12 or complements thereof.

The isolated nucleic acid molecules of the invention can be single-stranded or double-stranded, for instance, double-stranded DNA. The invention includes nucleic acid molecules containing modified nucleotides. For instance, the nucleic acid molecules of the invention can be created by introducing one or more nucleotide substitutions, additions or deletions into the corresponding HCV NS3 nucleotide sequence, such that one or more amino acid substitutions, additions or deletions are introduced into the encoded protein.

In another embodiment, an isolated nucleic acid molecule of the invention is at least about 10 nucleotides, about 12 nucleotides, about 15 nucleotides, about 18 nucleotides, about 20 nucleotides, or about 25 nucleotides in length and hybridizes under stringent conditions to the nucleic acid molecule comprising at least one NS3 nucleotide sequence.

Host cells and vectors for replicating the nucleic acid molecules and for expressing polypeptides are also provided. Any vectors or host cells may be used (e.g., prokaryotic or eukaryotic). Many vectors and host cells are known in the art for such purposes. It is well within the skill of the art to select an appropriate set for the desired application. In one embodiment, the vector is pcDNA3 and the host cells are 293-FS cells.

Techniques for isolating nucleic acid sequences encoding a NS3 protease domain using probe-based methods are conventional techniques and are well known to those skilled in the art. For example, the polymerase chain reaction (PCR) method disclosed by Mullis et al. (U.S. Pat. No. 4,683,195) and Mullis (U.S. Pat. No. 4,683,202), incorporated herein by reference, may be used. Probes for isolating such nucleic acid sequences may be based on published nucleic acid or protein sequences.

The sequence of an isolated NS3 nucleic acid (or polypeptide) can be compared to control NS3 sequences to determine the genotype of the HCV from which the nucleic acid was isolated. As known in the art, similarity between two polynucleotides or polypeptides is determined by comparing the nucleotide or amino acid sequence and its conserved nucleotide or amino acid substitutes of one polynucleotide or polypeptide to the sequence of a second polynucleotide or polypeptide. Also known in the art is “identity” which means the degree of sequence relatedness between two polypeptide or two polynucleotide sequences as determined by the identity of the match between two strings of such sequences. Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

While there exist a number of methods to measure identity and similarity between two polynucleotide or polypeptide sequences, the terms “identity” and “similarity” are well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo, H., and Lipman, D., SIAM J. Applied Math., 48: 1073 (1988). Methods commonly employed to determine identity or similarity between two sequences include, but are not limited to those disclosed in Guide to Huge Computers, Martin J. Bishop, ed., Academic Press, San Diego, 1994, and Carillo, H., and Lipman, D., SIAM J. Applied Math. 48:1073 (1988).

Preferred methods to determine identity are designed to give the largest match between the two sequences tested. Methods to determine identity and similarity are codified in computer programs. Computer program methods to determine identity and similarity between two sequences include, but are not limited to, GCG program package (Devereux, et al., Nucl. Acid Res. 12(1):387 (1984)), BLASTP, BLASTN, FASTA (Atschul, et al., J. Mol. Biol. 215:403 (1990)). The degree of similarity or identity referred to above is determined as the degree of identity between the two sequences, often indicating a derivation of the first sequence from the second. The degree of identity between two nucleic acid sequences may be determined by means of computer programs known in the art such as GAP provided in the GCG program package (Needleman and Wunsch, J. Mol. Biol. 48:443-453 (1970)). For purposes of determining the degree of identity between two nucleic acid sequences for the present invention, GAP can be used with the following settings: GAP creation penalty of 5.0 and GAP extension penalty of 0.3.

The invention further encompasses methods for producing a translated polypeptide of the invention using a HCV cassette cloned in a phenotype screening vector. In general terms, the production of a recombinant form of a protein typically involves the following steps.

A nucleic acid molecule is first obtained that encodes a NS3 protease domain and optionally, NS3 helicase and/or NS4A domains. The nucleic acid molecule is then placed in operable linkage with suitable control sequences as well as nucleic acids coding for part or all of the NS polyprotein (e.g., NS4B, NS5A) and a reporter molecule to form an expression unit containing the protein open reading frame. The expression unit is used to transform (i.e., transfect) a suitable host and the transformed host is cultured under conditions that allow the production of the recombinant polypeptide. Upon production of the polypeptide, the reporter molecule is cleaved from the polypeptide and secreted from the cell if the protease moiety of the polypeptide is functional.

Each of the foregoing steps can be accomplished in a variety of ways. For example, the construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier and are otherwise known to persons skilled in the art. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce a desired recombinant polypeptide.

A variety of expression systems may be used, including yeast, bacterial, animal, plant, eukaryotic and prokaryotic systems. In some embodiments, mammalian cell culture systems are preferred.

Vectors

Expression units for use in the present invention will generally comprise the following elements, operably linked in a 5′ to 3′ orientation: a transcriptional promoter operable in mammalian cells (e.g., a CMV promoter), a HCV cassette from a patient (i.e., a nucleic acid encoding NS3/4A or a nucleic acid encoding NS3 protease domain), a DNA sequence encoding at least one additional NS domain (e.g., NS3 helicase/4A/4B/5A (when using a cassette comprising NS3 protease) or NS4B/5A (when using a cassette comprising NS3/4A) optionally joined to a DNA sequence encoding 5B (e.g. the first 6 amino acids of 5B)), and a DNA sequence encoding a reporter (e.g., luciferase). As discussed above, any other arrangement of the HCV cassette and reporter fused to or within a nucleic acid encoding a HCV NS polyprotein portion may be used in the vectors of the invention. The selection of suitable promoters will be determined by the selected host cell and will be evident to one skilled in the art and are discussed more specifically below.

Mammalian expression vectors for use in carrying out the present invention will include a promoter capable of directing the transcription of the fusion protein. Preferred promoters include viral promoters and cellular promoters. Viral promoters include a CMV promoter, the major late promoter from adenovirus 2 (Kaufman and Sharp, Mol. Cell. Biol. 2: 1304-13199, 1982) and the SV40 promoter (Subramani et al., Mol. Cell. Biol. 1: 854-864, 1981). Cellular promoters include the mouse metallothionein 1 promoter (Palmiter et al., Science 222: 809-814, 1983) and a mouse V kappa (see U.S. Pat. No. 6,291,212) promoter (Grant et al., Nuc. Acids Res. 15: 5496, 1987). A particularly preferred promoter is a mouse V_{H} (see U.S. Pat. No. 6,291,212) promoter (Loh et al., ibid.).

Transformation

The phenotype screening vector and HCV cassette of the invention may be introduced into cultured mammalian cells by, for example, calcium phosphate-mediated transfection (Wigler et al., Cell 14: 725, 1978; Corsaro and Pearson, Somatic Cell Genetics 7: 603, 1981; Graham and Van der Eb, Virology 52:456, 1973.) Other techniques for introducing cloned DNA sequences into mammalian cells, such as electroporation (Neumann et al., EMBO J. 1: 841-845, 1982), or lipofection may also be used.

In order to identify cells that have integrated the cloned DNA, a selectable marker may be introduced into the cells along with the gene or cDNA of interest. Selectable markers for use in cultured mammalian cells include genes that confer resistance to drugs, such as neomycin, hygromycin, and methotrexate. The selectable marker may be an amplifiable selectable marker. An amplifiable selectable marker includes the DHFR gene (see U.S. Pat. No. 6,291,212). Selectable markers are reviewed by Thilly (Mammalian Cell Technology, Butterworth Publishers, Stoneham, Mass.) and the choice of selectable markers is well within the level of ordinary skill in the art.

Host Cells

The invention also includes a cell, preferably a mammalian cell transformed (i.e., transfected) to express a recombinant polypeptide of the invention (i.e., NS3/4A/4B/5A/reporter). In addition to the transformed host cells themselves, the present invention also includes a culture of those cells, preferably a monoclonal (clonally homogeneous) culture, or a culture derived from a monoclonal culture, in a nutrient medium. If the reporter peptide is secreted, the medium will contain the reporter peptide, with the cells, or without the cells if they have been filtered or centrifuged away.

Host cells for use in practicing the invention include eukaryotic cells, and in some cases prokaryotic cells, capable of being transformed or transfected with exogenous DNA and grown in culture, such as cultured mammalian, insect, fungal, plant and bacterial cells.

Host cells containing DNA constructs of the present invention are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals and growth factors. The growth medium will generally select for cells containing the DNA construct by, for example, drug selection or deficiency in an essential nutrient which is complemented by the selectable marker on the DNA construct or co-transfected with the DNA construct.

Cultured mammalian cells are generally grown in commercially available serum-containing or serum-free media. Selection of a medium appropriate for the particular cell line used is within the level of ordinary skill in the art. Transfected mammalian cells are allowed to grow for a period of time, typically 1-2 days, to begin expressing the DNA sequence(s) of interest. Drug selection is then applied to select for growth of cells that are expressing the selectable marker in a stable fashion. For cells that have been transfected with an amplifiable selectable marker the drug concentration may be increased in a stepwise manner to select for increased copy number of the cloned sequences, thereby increasing expression levels.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

### EXAMPLES

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### Example 1

### HCV NS3 Domain Degenerate Primer Design

Primers preferably capable of annealing to both HCV genotypes 1a and 1b were designed to amplify the HCV NS3 region. To design the primers, published HCV Genotype 1 (mostly 1a and 1b) sequences were collected and aligned to identify regions with a high degree of homology. The appearance frequency of each nucleic acid within a homology region was calculated. A cut-off frequency percentage can be assigned to keep the degeneracy of the designed primer in an acceptable range. Another considering factor in designing the degenerate primer is to try to keep the 5 nucleic acids at most 3′ end free of degeneracy.

### Example 2

### Amplifying HCV NS3 Domain

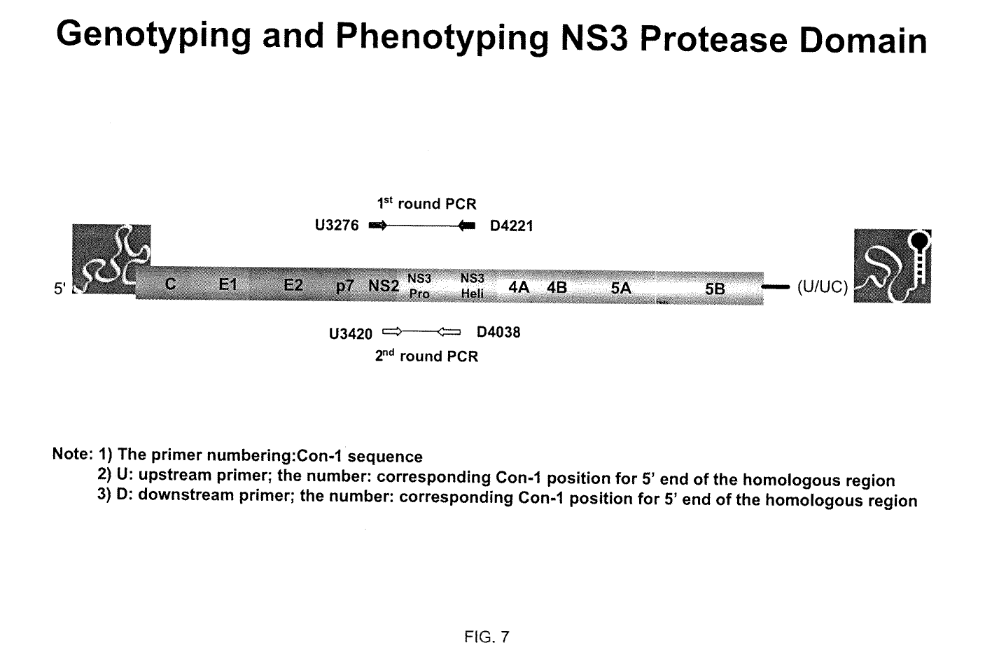

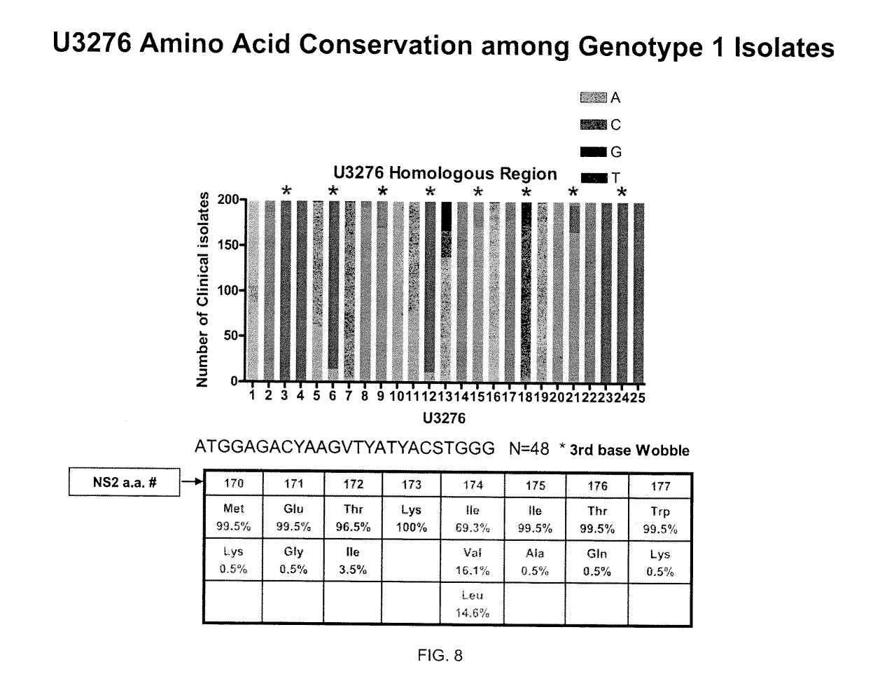

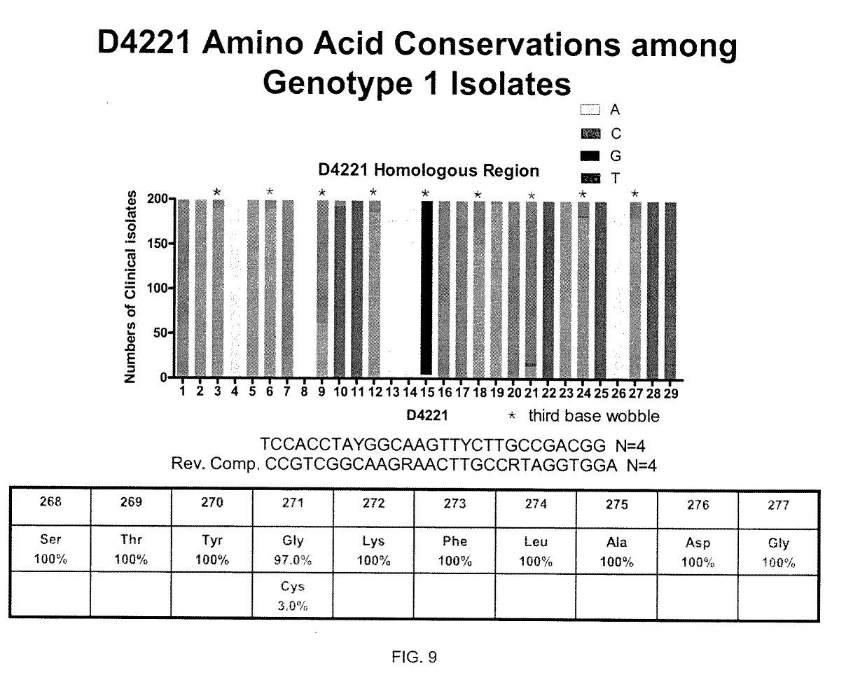

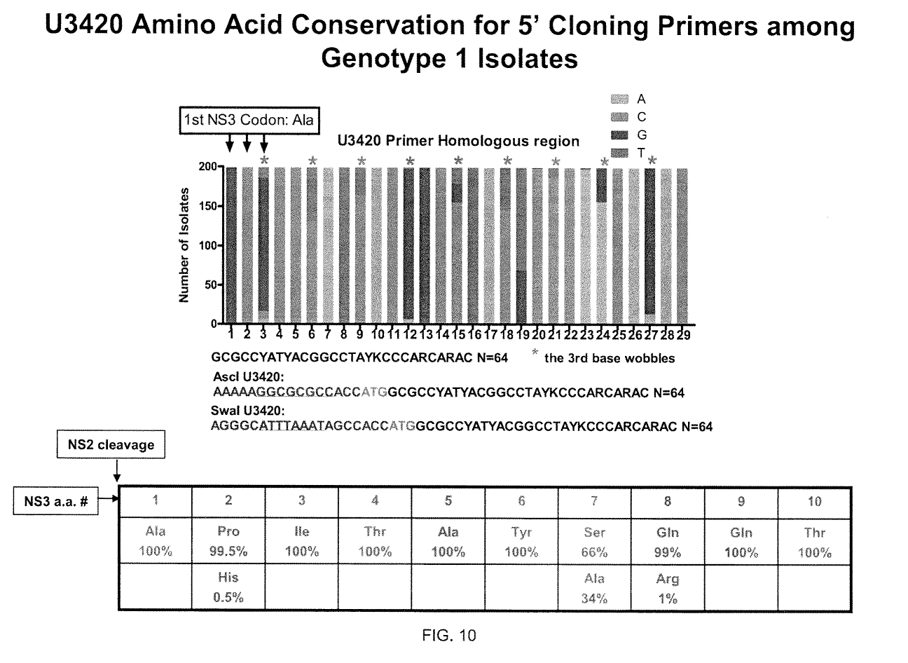

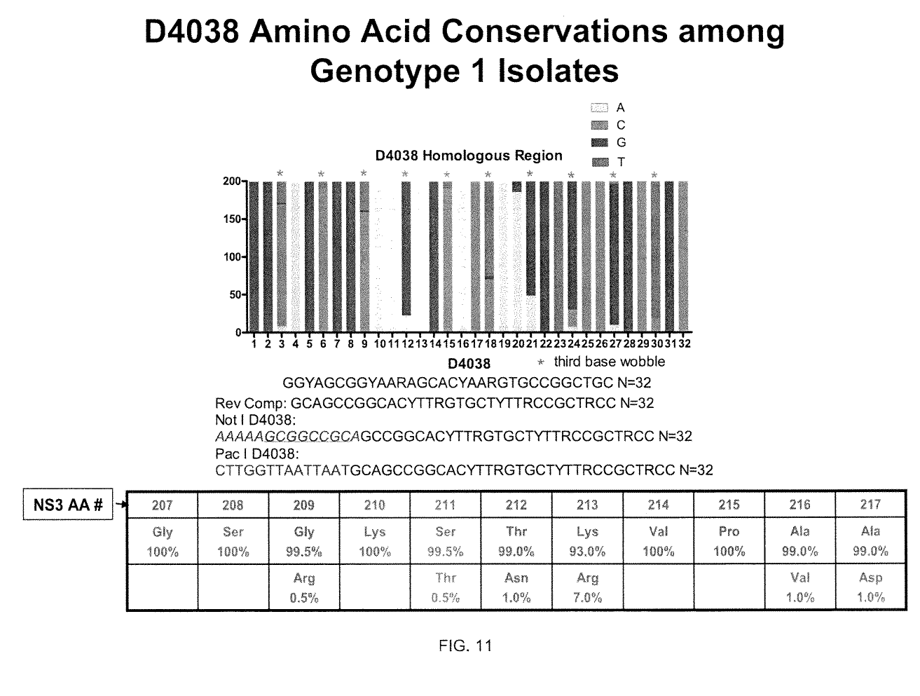

Viral RNA was isolated from clinical samples and the HCV NS3 domain was amplified using first and second round degenerate primers. The region of HCV amplified is shown in FIG. 7. FIG. 7 shows the regions where reasonable homology was identified. As indicated, “U” represents an upstream primer and “D” represents a downstream primer, with the numbers corresponding to the Con-1 position for the 5′ end of the homologous region. The second round primers, U3420 and D4038, carry restriction sites for cassette cloning into a phenotyping vector. U3420 also has the start codon and kozac sequence for protein translation. FIGS. 8-11 show the amino acid conservation among genotype 1 isolates in primers U3276, D4221, U3420, and D4038, respectively.

### Example 3

### HCV Genotype Testing with Clinical Samples

Clinical isolates were obtained from multiple sources including hospital, clinical lab and commercial entities.

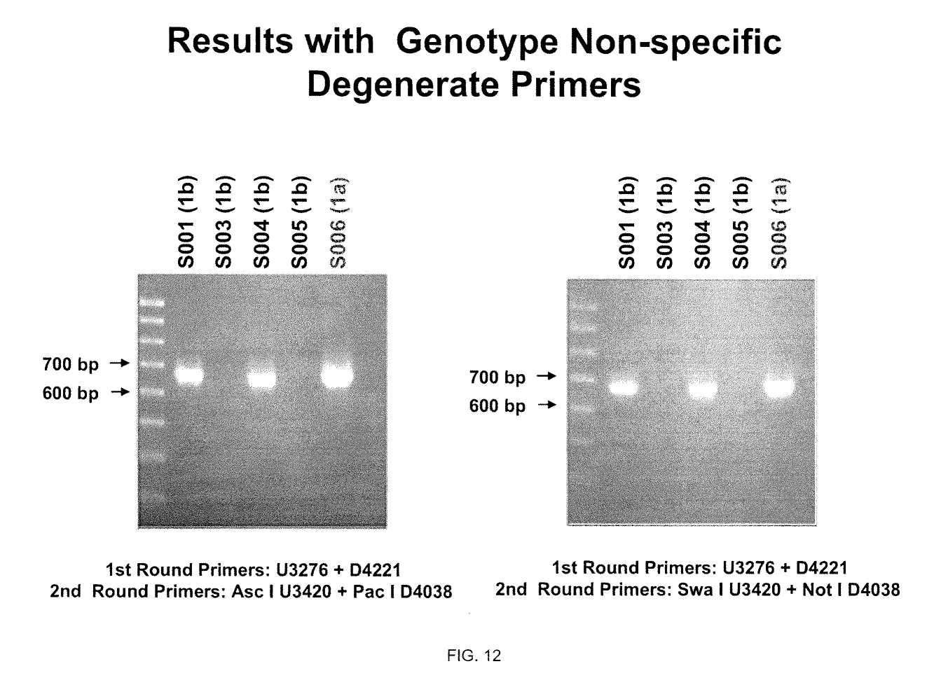

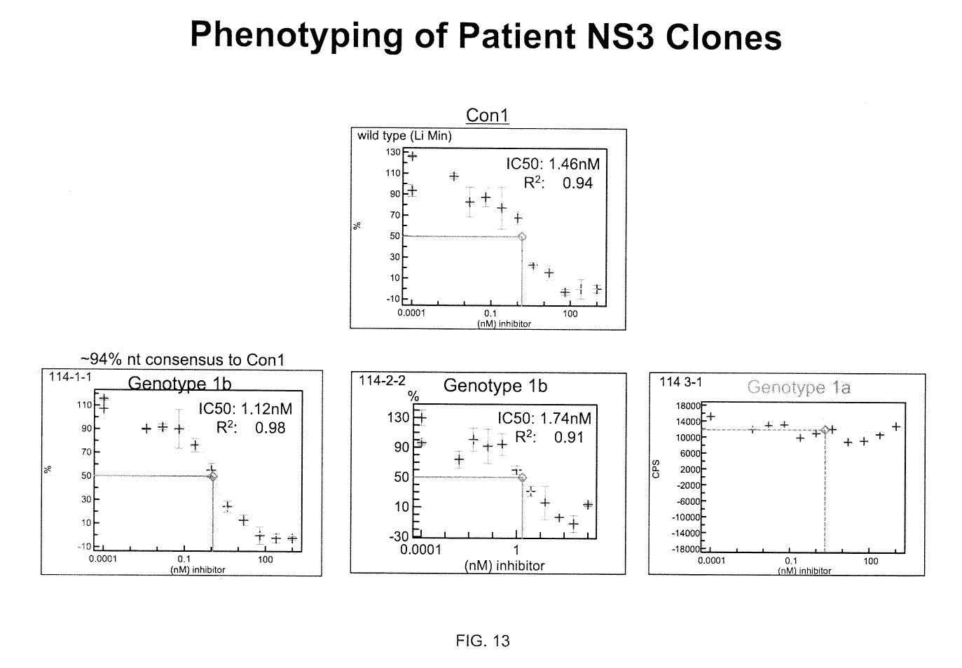

The results of phenotyping patient NS3 clones is shown in FIG. 13. The three amplified clinical samples shown in FIG. 12 were cloned into the phenotyping vector and characterized by phenotyping assay.

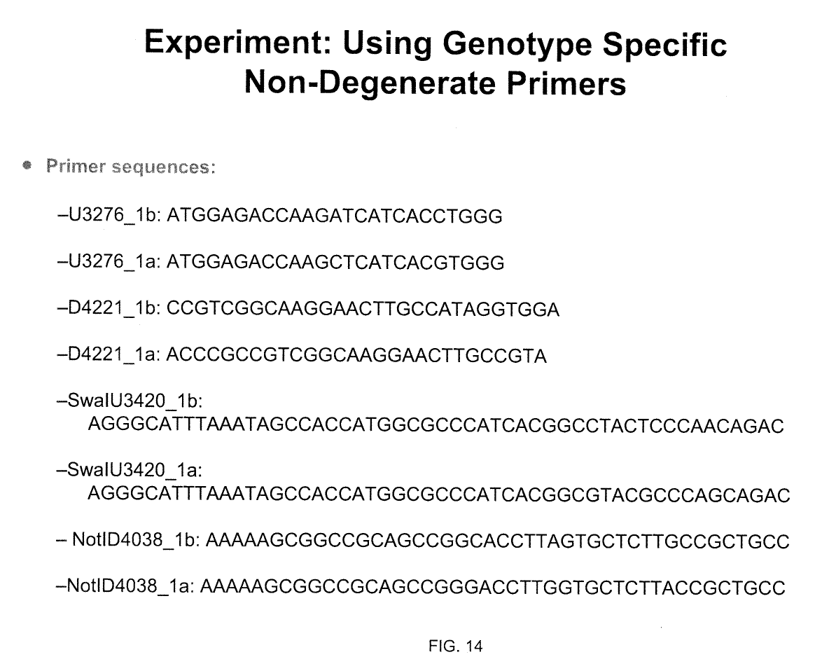

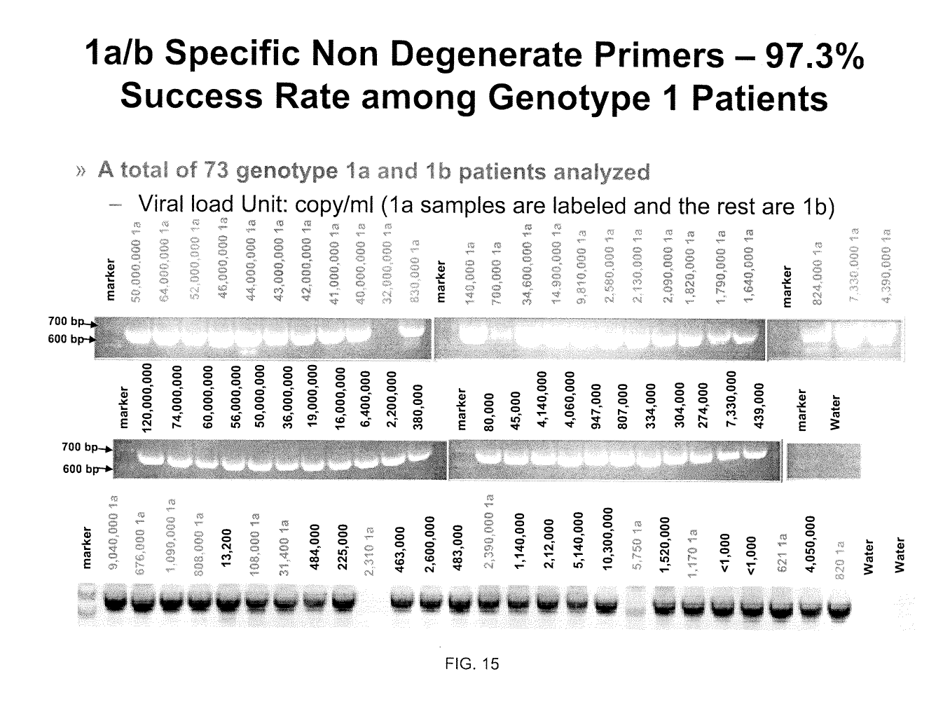

FIG. 14 shows the sequences of genotype 1a/b specific non-degenerate primers. FIG. 15 shows the products that resulted from PCR amplification using genotype 1a/b specific non-degenerate primers. The PCR products were cleaned and the population was sequenced. Sequencing results revealed that they are HCV 1a or 1b sequences.

### Example 4

### HCV Phenotyping Assay

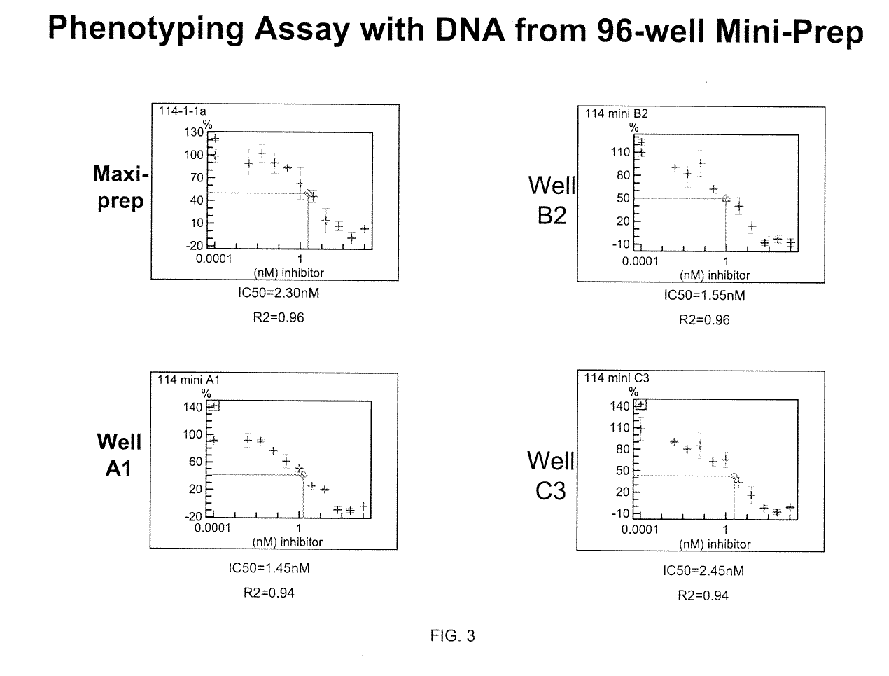

FIG. 3 shows a phenotyping assay with DNA from a 96-well mini-prep.

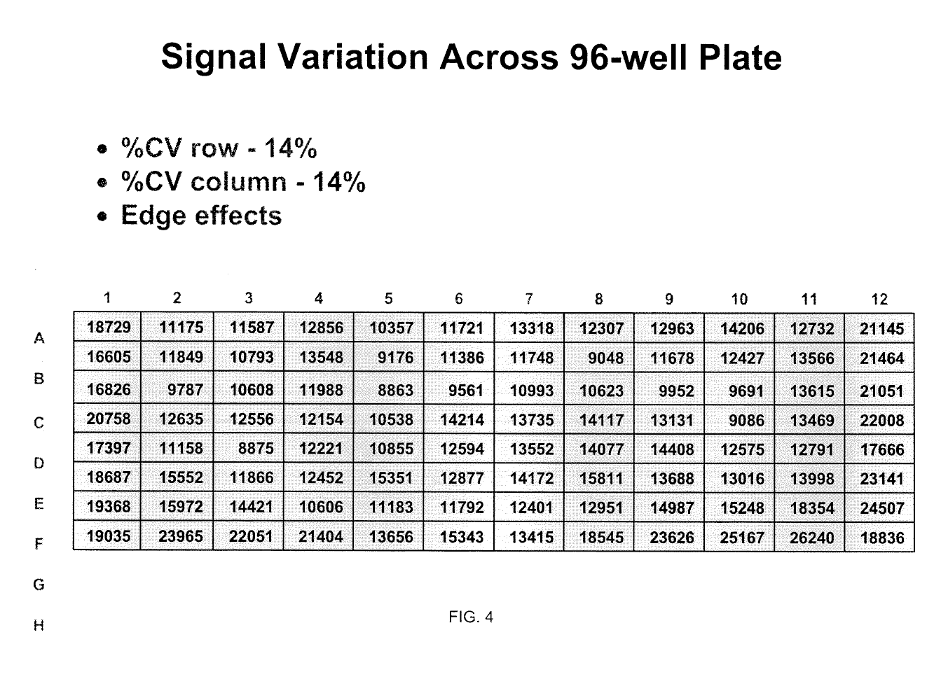

FIG. 4 shows the signal variation across the 96-well plate in high throughput robotic system.

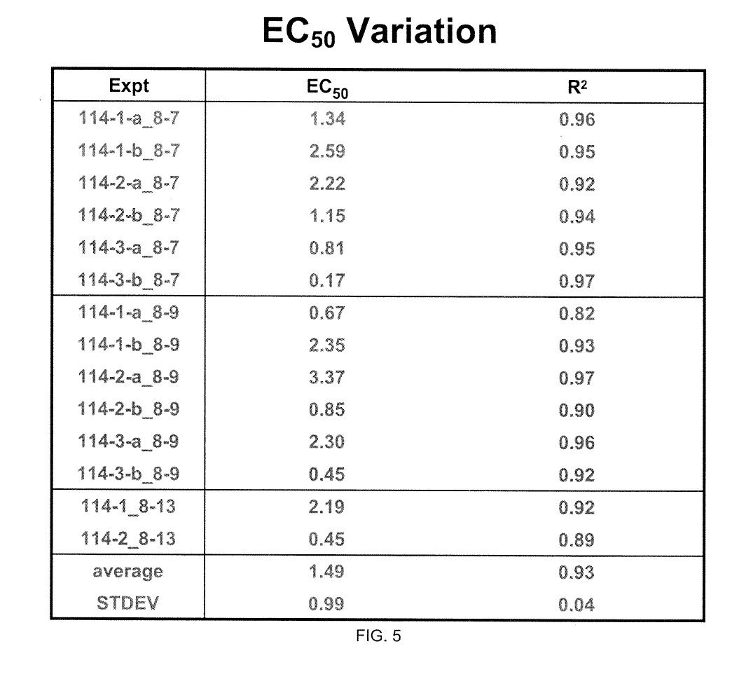

FIG. 5 shows the EC₅₀ variation with the same phenotyping vector intra-day and inter-day.

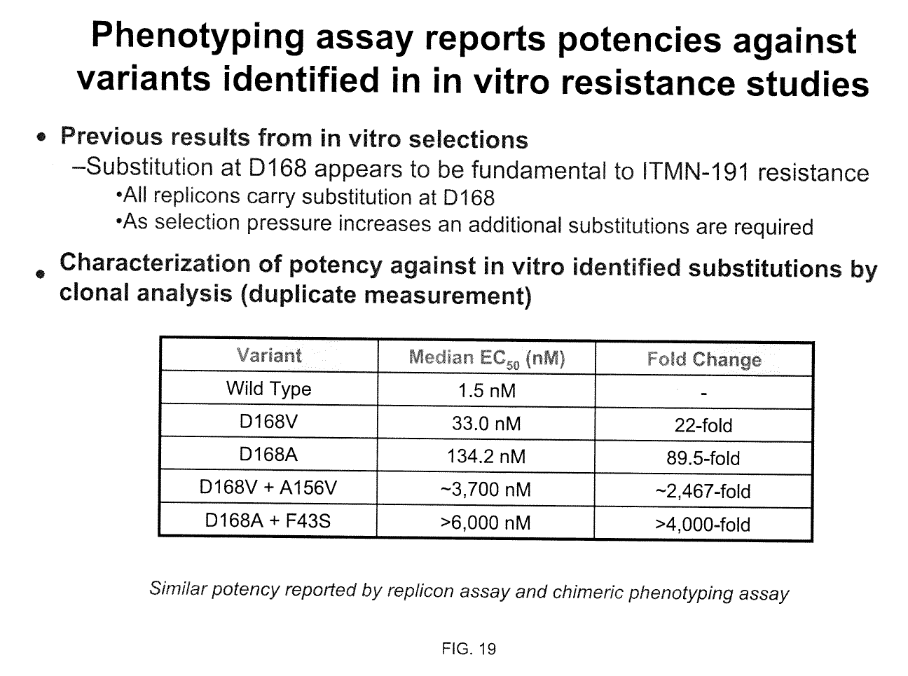

FIG. 19 shows the phenotyping EC₅₀ of NS3 variants that had been identified in the in vitro HCV replicon resistance studies. The individual EC50 value of each variant and the EC50 rank order of different variants correlate well with the replicon transient transfection data.

### Example 5

### Automation of HCV Phenotyping Assay

The cell based phenotyping assay can be adapted for a high throughput system (HTS). For instance, using the assay, it is possible to screen at least 96 sequences at 5 time points in 40 patients, i.e., 19,200 targets can be characterized within a 10 month period.

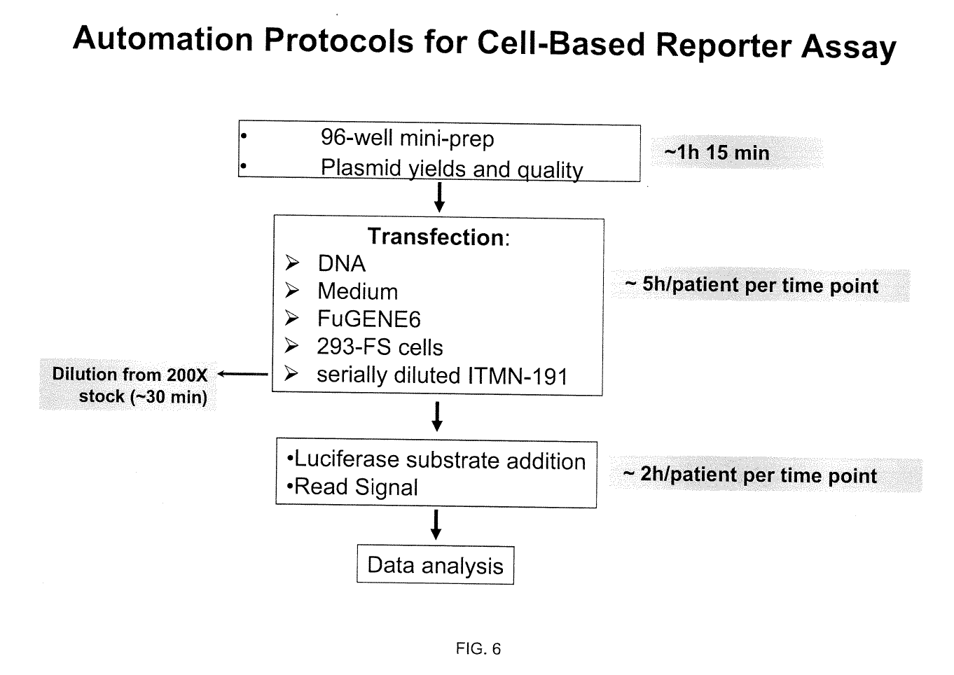

FIG. 6 shows a flow chart depicting exemplary steps of an automation protocol for a cell-based reporter assay. DNA obtained from the 96-well mini-prep described in Example 1 can be used in the assay. The DNA can be transfected into cells that are subsequently treated with an inhibitor of NS3/4A protease activity such as serially-diluted ITMN-191. The substrate for the reporter secreted luciferase is added and the activity of secreted luciferase is determined. Secreted luciferase activity is indicative of the protease activity, which in turn is indicative of the phenotype of the HCV.

Although the invention has been described with reference to the presently preferred embodiment, it should be understood that various changes and modifications, as would be obvious to one skilled in the art, can be made without departing from the spirit of the invention. Accordingly, the invention is limited only by the following claims.

We claim:

1. A population of first round upstream primers, wherein each primer comprises a nucleic acid sequence encoding Met/Lys-Glu/Gly-Thr/Ile-Lys-Ile/Val/Leu-Ile/Ala-Thr/Gln-Trp/Lys.

2. The population of primers of claim 1, wherein each primer encodes an amino acid sequence and wherein a population of said amino acid sequences has the following distribution with respect to each amino acid:
Met (99.5%)/Lys (0.5%)-Glu (99.5%)/Gly (0.5%)-Thr (96.5%)/Ile (3.5%)-Lys (100%)-Ile (69.3%)/Val (16.1%)/Leu (14.6%)-Ile (99.5%)/Ala (0.5%)-Thr (99.5%)/Gln (0.5%)-Trp (99.5%)/Lys (0.5%)

3. The population of primers of claim 1, wherein each primer comprises a nucleic acid sequence of ATGGAGACYAAGVTYATYACSTGGG, wherein Y is C or T, V is A or G or C, and S is G or C.

4. The population of primers of claim 1, wherein each primer comprises a nucleic acid sequence of ATGGAGACYAMAMGVTYAMTYAMCSTGGG, wherein Y is C or T, V is A or G or C, and S is G or C, and wherein AM is a modified adenosine.

5. The population of primers of claim 1, wherein each primer comprises a nucleic acid sequence of AMTGMGMAGACYAMAMGVTYAMTYAMCMSTGGG, wherein Y is C or T, V is A or G or C, and S is G or C, and wherein AM is a modified adenosine and GM is a modified guanosine.

6. The population of primers of claim 1, wherein the population comprises at least 1 primer.

7. A population of first round downstream primers, wherein the complement of each primer comprises a nucleic acid sequence encoding Ser-Thr-Tyr-Gly/Cys-Lys-Phe-Leu-Ala-Asp-Gly.

8. The population of primers of claim 7, wherein the complement of each primer encodes an amino acid sequence and wherein a population of said amino acid sequences has the following distribution with respect to each amino acid:
Ser (100%)-Thr (100%)-Tyr (100%)-Gly (97.0%)/Cys (3.0%)-Lys (100%)-Phe (100%)-Leu (100%)-Ala (100%)-Asp (100%)-Gly (100%).

9. The population of primers of claim 7, wherein each primer comprises a nucleic acid sequence of CCGTCGGCAAGRAACTTGCCRTAGGTGGA, wherein R is A or G.

10. The population of primers of claim 7, wherein each primer comprises a nucleic acid sequence of CCGTCGGCAAGRAMACTTMGCCRTMAGGTMGGA, wherein R is A or G, and wherein AM is a modified adenosine, TM is a modified thymidine.

11. The population of primers of claim 7, wherein each primer comprises a nucleic acid sequence of CCGTMCGGCAAMGRAMACTMTMGCCRTMAMGGTMGGA, wherein R is A or G, and wherein AM is a modified adenosine, TM is a modified thymidine.

12. The population of primers of claim 7, wherein the population comprises at least 1 primer.

13. A population of second round upstream primers, wherein each primer comprises a nucleic acid sequence encoding Ala-Pro/His-Ile-Thr-Ala-Tyr-Ser/Ala-Gln/Arg-Gln-Thr.

14. The population of primers of claim 13, wherein each primer encodes an amino acid sequence and wherein a population of said amino acid sequences has the following distribution with respect to each amino acid:
Ala (100%)-Pro (99.5%)/His (0.5%)-Ile (100%)-Thr (100%)-Ala (100%)-Tyr (100%)-Ser (66%)/Ala (34%)-Gln (99%)/Arg (1%)-Gln (100%)-Thr (100%).

15. The population of primers of claim 13, wherein each primer comprises a nucleic acid sequence of GCGCCYATYACGGCCTAYKCCCARCARAC, wherein Y is C or T, K is G or T, and R is A or G.

16. The population of primers of claim 13, wherein each primer comprises a nucleic acid sequence of GCGCCYAMTMYACGGCMCTMAMYKCCCMARCMAMRAC, wherein Y is C or T, K is G or T, and R is A or G and wherein AM is a modified adenosine, CM is a modified cytidine, and TM is a modified thymidine.

17. The population of primers of claim 13, wherein each primer comprises a nucleic acid sequence of GCGCCYAMTMYACGGCCTAMYKCCCARCAMRAC, wherein Y is C or T, K is G or T, and R is A or G and wherein AM is a modified adenosine, and TM is a modified thymidine.

18. The population of primers of claim 13, wherein each primer comprises a nucleic acid sequence of AGGGCATTTAAATAGCCACCATGGCGCCYATYACGGCCTAYKCCCARCARAC, wherein Y is or T, K is G or T, and R is A or G.

19. The population of primers of claim 13, wherein each primer comprises a nucleic acid sequence of AAAAAGGCGCGCCACCATGGCGCCYATYACGGCCTAYKCCCARCARAC, wherein Y is C or T, K is G or T, and R is A or G.

20. The population of primers of claim 13, wherein the population comprises at least 1 primer.

21. A population of second round downstream primers, wherein the complement of each primer comprises a nucleic acid sequence encoding Gly-Ser-Gly/Arg-Lys-Ser/Thr-Thr/Asn-Lys/Arg-Val-Pro-Ala/Val-Ala/Asp.

22. The population of primers of claim 21, wherein the complement of each primer encodes an amino acid sequence and wherein a population of said amino acid sequences has the following distribution with respect to each amino acid:
Gly (100%)-Ser (100%)-Gly (99.5%)/Arg (0.5%)-Lys (100%)-Ser (99.5%)/Thr (0.5%)-Thr (99%)/Asn (1%)-Lys (93%)/Arg (7%)-Val (100%)-Pro (100%)-Ala (99%)/Val (1%)-Ala (99%)/Asp (1%)

23. The population of primers of claim 21, wherein each primer comprises a nucleic acid sequence of GCAGCCGGCACYTTRGTGCTYTTRCCGCTRCC, wherein Y is C or T and R is A or G.

24. The population of primers of claim 21, wherein each primer comprises a nucleic acid sequence of GCAGCCGGCAMCYTTMRGTMGCTMYTMTMRCMCGCTMRCC, wherein Y is C or T and R is A or G, and wherein AM is a modified adenosine, TM is a modified thymidine, and CM is a modified cytidine.

25. The population of primers of claim 21, wherein each primer comprises a nucleic acid sequence of GCAGCCGGCACYTTMRGTGCTMYTTMRCCGCTMRCC, wherein Y is C or T and R is A or G, and wherein TM is a modified thymidine.

26. The population of primers of claim 21, wherein each primer comprises a nucleic acid sequence of AAAAAGCGGCCGCAGCCGGCACYTTRGTGCTYTTRCCGCTRCC, wherein Y is C or T and R is A or G.

27. The population of primers of claim 21, wherein each primer comprises a nucleic acid sequence of CTTGGTTAATTAATGCAGCCGGCACYTTRGTGCTYTTRCCGCTRCC, wherein Y is C or T and R is A or G.

28. The population of primers of claim 21, wherein the population comprises at least 1 primer.

29. A kit comprising the primers of claim 1 and claim 7.

30. A kit comprising the primers of claim 13 and claim 21.

31. A method of amplifying HCV NS3 protease domain from a sample of a patient infected or suspect of being infected with HCV comprising amplifying a nucleic acid sample from said sample using the primers of claim 1 and claim 7 or claim 13 and claim 21 or a combination thereof.

32. A method of determining the genotype of a HCV virus comprising amplifying a fragment within the protease domain of the HCV virus and determining the genotype of the HCV virus based on the genotype of said fragment.

33. The method of claim 32, wherein the fragment is amplified by using the primers of claim 1 and claim 7 or claim 13 and claim 21 or a combination thereof.

34. A method of determining the presence of a drug resistant HCV virus comprising conducting amplification of a fragment within the protease domain of a HCV from a HCV sample, determining the presence of a mutation associated with drug resistance within the fragment, wherein the presence of the mutation is indicative of the presence of drug resistant HCV.

35. The method of claim 34, wherein amplification is conducted using the primers of claim 1 and claim 7 or claim 13 and claim 21 or a combination thereof.

36. A method of determining the phenotype of a HCV virus comprising cloning NS3 protease domain of the HCV virus into a screening vector comprising a polynucleotide encoding HCV NS3Helicase, 4A, 4B, 5A and a secreted luciferase reporter, wherein the polynucleotide encoding the NS3 protease domain, NS3 Helicase, 4A, 4B, 5A and the secreted luciferase reporter are operably linked so that the presence of a functional NS3 protease domain is indicated by the secretion of the secreted luciferase reporter.

37. The method of claim 36, wherein the polynucleotide encodes HCV NS3Helicase, 4A, 4B, 5A, the first 6 amino acids of 5B and a secreted luciferase reporter.

38. A screening vector comprising a polynucleotide encoding HCV NS3 Helicase, 4A, 4B, 5A, and a secreted luciferase reporter, operably linked so that insertion of a functional NS3 protease domain in the screening vector is indicated by the secretion of the secreted luciferase reporter.

39. The screening vector of claim 38, wherein the polynucleotide encodes HCV NS3 Helicase, 4A, 4B, 5A, the first 6 amino acids of 5B and a secreted luciferase reporter.

40. A set of primers comprising an upstream primer comprising a sequence selected from the group consisting of ATGGAGACCAAGATCATCACCTGGG and ATGGAGACCAAGCTCATCACGTGGG, and a down stream primer comprising a sequence selected from the group consisting of CCGTCGGCAAGGAACTTGCCATAGGTGGA and ACCCGCCGTCGGCAAGGAACTTGCCGTA.

41. A set of primers comprising an upstream primer comprising a sequence selected from the group consisting of AGGGCATTTAAATAGCCACCATGGCGCCCATCACGGCCTACTCCCAACAGAC and AGGGCATTTAAATAGCCACCATGGCGCCCATCACGGCGTACGCCCAGCAGAC, and a downstream primer comprising a sequence selected from the group consisting of AAAAAGCGGCCGCAGCCGGCACCTTAGTGCTCTTGCCGCTGCC and AAAAAGCGGCCGCAGCCGGGACCTTGGTGCTCTTACCGCTGCC.

42. A primer comprising a sequence selected from the group consisting of ATGGAGACCAAGATCATCACCTGGG, ATGGAGACCAAGCTCATCACGTGGG, CCGTCGGCAAGGAACTTGCCATAGGTGGA, ACCCGCCGTCGGCAAGGAACTTGCCGTA, AGGGCATTTAAATAGCCACCATGGCGCCCATCACGGCCTACTCCCAACAGAC, AGGGCATTTAAATAGCCACCATGGCGCCCATCACGGCGTACGCCCAGCAGAC, AAAAAGCGGCCGCAGCCGGCACCTTAGTGCTCTTGCCGCTGCC, and AAAAAGCGGCCGCAGCCGGGACCTTGGTGCTCTTACCGCTGCC.
